(19) United States
(12) Patent Application Publication
(10) Pub. No.: US 2018/0353748 A1
(43) Pub. Date: Dec. 13, 2018

(54) REVERSE IONTOPHORESIS BIOSENSING WITH REDUCED SAMPLE VOLUMES

(71) Applicants: University Of Cincinnati, Cincinnati, OH (US); Eccrine Systems, Inc., West Chester Township, OH (US)

(72) Inventors: Jason C. Heikenfeld, Cincinnati, OH (US); Andrew Jajack, Cincinnati, OH (US); Elizabeth Sheetz, Cincinnati, OH (US)

(73) Assignee: University Of Cincinnati, Cincinnati, OH (US)

(21) Appl. No.: 15/747,599

(22) PCT Filed: Jul. 25, 2016
(86) PCT No.: PCT/US16/43862

§ 371 Date: Jan. 25, 2018

Related U.S. Application Data
(60) Provisional application No. 62/196,541, filed on Jul. 24, 2015.
(60) Provisional application No. 62/328,907, filed on Apr. 28, 2016.
(60) Provisional application No. 62/357,643, filed on Jul. 1, 2016.

(51) Int. Cl. A61N 1/04; A61N 1/36; A61B 5/145

(52) U.S. Cl. CPC A61N 1/0444; A61N 1/36031; A61B 5/14546; A61B 5/14514; A61B 5/14521

(57) ABSTRACT

A device for sensing biofluid placed on skin with at least one pre-existing pathway includes a first analyte-specific sensor for sensing a first analyte in the biofluid and a volume-reduced pathway between skin and the first analyte-specific sensor configured to allow an advective flow of the biofluid from the at least one pre-existing pathway toward the first analyte-specific sensor. The first analyte-specific sensor does not consume the first analyte. The device further includes an iontophoresis electrode and a counter electrode for bringing the first analyte into the at least one pre-existing pathway. The biofluid may be more than 50% interstitial fluid or more than 50% sweat. The device may also include at least one of a wicking collector, a wicking coupler, or a wicking pump.

### CROSS-REFERENCE TO RELATED APPLICATIONS

This application claims priority to U.S. Provisional Application Nos. 62/196,541 filed Jul. 24, 2015, 62/328,907 filed Apr. 28, 2016, and 62/357,643 filed Jul. 1, 2016, the disclosures of which are hereby incorporated by reference herein in their entirety.

### BACKGROUND OF THE INVENTION

Non-invasive biosensing technologies have enormous potential for applications ranging from athletics, to neonatology, to pharmacological monitoring, to personal digital health, to name a few applications. The sweat ducts can provide a route of access to many of the same biomarkers, chemicals, or solutes that are carried in blood and can provide significant information enabling one to diagnose ailments, health status, toxins, performance, and other physiological attributes even in advance of any physical sign. Sweat has many of the same analytes and analyte concentrations found in blood and interstitial fluid. Interstitial fluid has even more analytes nearer to blood concentrations than sweat does, especially for larger sized and more hydrophilic analytes (such as proteins).

If biofluid access through the skin has such significant potential as a sensing paradigm, then why has it not emerged beyond decades-old usage in infant chloride sweat assays for Cystic Fibrosis or in illicit drug monitoring patches? Or, why for example did past reverse iontophoresis products for interstitial fluid extraction dominantly through eccrine ducts, such as GlucoWatch, also fail commercially? In part, past challenges and failures have been due to the difficulty of finding ergonomic and acceptable ways to generate the biofluid for sampling (non-invasive, continuous, non-irritating, etc.). In part, past challenges and failures have also been due to the difficulty of obtaining an adequate volume of sample for a measurement of analytes in these types of biofluids. Reducing the sample volume is critical for fast sampling times and/or to allow lower sample generation rates (e.g., less reverse iontophoresis current and related stress on skin). However, simply reducing the sample volume, especially for the case of reverse-iontophoresis, brings about secondary challenges such as pH changes.

A more detailed background description is now provided, beginning here with a discussion of fluid sampling rate. Assume a case where sweat glands dominantly provide the pre-existing pathways. Next, using information from Cunningham in Chapter 7 of the book In Vivo Glucose Sensing, 2010, assume a device having a sampling area of 1 cm² applied on a person's wrist. Assuming use of a sweat gland density of 150/cm² for the wrist, a sensor that is 0.55 cm in radius (1.1 cm in diameter) would cover about 1 cm² area or approximately 150 sweat glands. Now, consider an example sample generation rate by reverse iontophoresis based on 15-150 nL of interstitial fluid being generated with 3 minutes of reverse iontophoresis at 0.3 mA/cm². Therefore, roughly 5 to 50 nL is generated per minute. Assuming a 1 cm² area for reverse iontophoresis, then the sample generation rate for the wrist would therefore be roughly 0.03 to 0.3 nL/min/gland. If the fluidic portion of the 1 cm² device is 127 μm thick (i.e., same as the gel used with the GlucoWatch), then fluidic volume is 12,700 nL. If that volume were to be completely filled with new interstitial fluid, it would require 2822 to 282 minutes (47 to 4.7 hours), which represents a very slow sampling interval. Using another estimate by Pikal and Shah “Transport mechanisms in iontophoresis. I. A theoretical model for the effect of electroosmotic flow on flux enhancement in transdermal iontophoresis” 1990, the sample generation rate of interstitial fluid is 6-19 μL/hr/mA, or about 12.5 μL/hr/mA*1 hr/60 min=0.21 μL/mA/min. For the above case of 0.3 mA/cm², the sample generation rate would be 63 nL/min/cm² which for 150 glands/cm² would be 0.42 nL/min/gland (higher than Cunningham's example, but still requiring greater than 2 hours for the sampling interval). Discrepancies between these numbers could be due to interpretations of volumes based on diluted analyte concentration, or non-ideal factors.

Next, consider the impact of the choice of sensing modality. The actual sampling interval and the time required to sense an analyte varies widely based on the method of sensing the analyte. For example, some sensors consume the sensed analyte (e.g., glucose and enzymatic/amperometric sensing) while others do not consume the analyte and respond by equilibrating to the local concentration of the analyte (e.g., ionselective or electrochemical aptamer-based sensors). An aptamer-based sensor may bind an analyte, but the analyte is not consumed (i.e., once the analyte binds, the same site will not bind further analyte, and the analyte can be released back into solution). Because sensors that consume the analyte do not require a complete refreshing of the sample volume (e.g., new sample replaces or washes away the old sample), sensors that consume the analyte will not apply to how sampling rate and sampling interval are described and calculated herein. Conversely, sensors that do not consume the analyte, therefore, will only respond as quickly as the sample volume is refreshed across the sensor.

Consider an example involving an electrochemical aptamer sensor for vasopressin. Assume the sensor is configured with a linear range of detection centered around vasopressin's normal concentration range in interstitial fluid, where the fluid is extracted by reverse iontophoresis. Unlike an amperometric sensor that consumes the analyte, the aptamer sensor does not consume the vasopressin, nor does the aptamer sensor aggregate detection of vasopressin over time. Therefore, the vasopressin must remain within the detection range if the sensor is to continue to detect vasopressin. The sampling interval for vasopressin, accordingly, would be in the multiple-hour range using a device with slow interstitial fluid refresh rate, such as those described in the above example. For detecting and protecting against dehydration, and other time-sensitive applications, such sampling intervals could be entirely too slow. Cortisol awakening response, for example, occurs within a 30 minute window and requires multiple readings during that window. The multi-hour sampling intervals mentioned above would be entirely too slow for such an application.

Consider instead an interstitial fluid wicking component that is composed of 5 μm deep channels that comprise 5% of the wicking component surface area, resulting in an interstitial fluid volume of at least 5E-4 cm*0.05*1 cm²=2.5E-5 mL or 25 nL. That is a roughly 500× lower sample volume. Such a substantially reduced sample volume can provide one or more significant advances in performance, such as: (1) greatly reduced sampling intervals (e.g., as fast as minutes even for sensors that do not consume the analyte); and (2) greatly reduced current density requirements for reverse iontophoresis. However, reducing the sample volume creates at least one secondary challenge, namely pH changes caused by water electrolysis.

Consider an illustrative example that assumes a 0.3 nL/min/gland interstitial fluid sample generation rate for 0.3 mA/cm² applied to skin. Assume only needing to fill the dermal duct of the eccrine sweat gland which has a diameter of about 15 μm and a length of about 2000 μm. This ductal volume is therefore 2000*3.14*7.5² μm³=0.35 nL. At 0.3 nL/min/gland it would require approximately 1 minute to get a fresh interstitial fluid sample to the skin surface (and 30 seconds for 0.6 nL/min/gland). This is likely at least one reason why GlucoWatch applies reverse iontophoresis for a period of 3 minutes followed by 7 minutes to allow glucose to diffuse into the gel and be sensed (such that it is not pulled back into skin during the subsequent application of voltage in the opposite polarity).

Further, assume the device covers skin having 100 glands/cm², where the device area is 1 cm² and a volume to be filled of 1 μL (the space between the device and skin to be filled is 10 μm thick). It would require 30 minutes to fill this volume at only 0.3 nL/min/gland, and 15 minutes at 0.6 nL/min/gland. If the system is starting at a pH of 7 for the 10 μm thick fluid volume, with reverse iontophoresis durations of 1 min, 10 min, and 30 min, the pH under the negative voltage electrode would drop to extreme levels, i.e., 0.7, 0.3, and −0.7, respectively (first order calculations only). Such pH changes can be impractical from both a skin safety perspective and from a biosensing perspective (e.g., they could degrade numerous types of analytes). Some interesting conclusions can be made. First, using reverse iontophoresis for sample generation of only interstitial fluid, with such small sample volumes, can be impractical, unless significant steps are taken to buffer the pH changes. Therefore in some cases, dilution of the interstitial fluid in sweat could be advantageous as it would also dilute the pH change. Second, for an example like this where the interstitial fluid is transported away and replenished into the sample volume, increasing the sample volume does not reduce the pH change (because doubling the sample volume dilutes the pH change, but also requires 2× more current to fill that sample volume, resulting in the same pH change as before). Third, where the interstitial fluid is transported away and replenished into the sample volume, the pH is theoretically constant for a given sample volume (is irrespective of current density), because both sample generation rate and current density scale linearly.

Next, an erroneous argument could be made that using a scheme where one continually reverses the voltage (like that used by GlucoWatch) could solve all issues with pH. Basically, each time voltage was applied the pH would advantageously start at the opposite end of the pH spectrum (e.g., would be modulated back and forth so the net pH would instead oscillate closer to a neutral pH of 7). However, that assumes the interstitial fluid sample stays in place, which as previously described is accurate for a technology such as GlucoWatch (glucose sensing, thick gel full of fluid), but not for a technology with a greatly reduced sample volume and a net transport of fluid away from the skin to at least one sensor. For example, if it requires 30 minutes to fill the volume and that volume is continually being emptied and taken to a sensor as quickly as it is generated, then the fluid volume is actually even lower (is being depleted) which makes the pH changes even greater. Also, if the fluid were not transported away, then a portion of the fluid could be pulled back into the body by reverse iontophoresis as well. Again, this is not a problem for technology like GlucoWatch (glucose sensing) but is a challenge for other types of devices.

It is also possible, at a cost of reduced reverse iontophoresis current density, to mitigate pH issues by using voltage at the electrodes (i.e., between the electrodes and solution) that is below the electrolysis potential for water: H₂O→½ O₂(g)+2 H⁺+2 e⁻ for +1.23 V at the anode, and 2 H₂O+2 e⁻→H₂(g)+2 OH⁻ for −0.83 V at the cathode. Diamond electrodes can extend this voltage threshold to as much as about 2V. A situation with a lower resistance across the skin (e.g., active sweating stimulated by carbachol iontophoresis) would therefore have reduced total voltage required for reverse iontophoresis. The voltage drop for a system on skin would be in part at the electrodes, in part across the skin, and in part across the tissue/body beneath the skin. For example, consider on the sweating forearm 0.2 mA applied with 5-10 V with a smallest electrode area of 0.95 cm² for a current density of about 0.2 mA/cm². Also, consider sweating under a Vitrode-J electrodes of 40 mm diameter placed 3 cm apart also on the forearm which registers a conductance of 100 μS, which for 10 V translates to 1 mA per 12 cm² or about 0.1 mA/cm². To be absolutely certain that the voltage across both electrodes was less than 2V (near the point of no electrolysis), based on first-order calculations, the current densities would need to be reduced from about 0.1 mA/cm² to about 0.05 mA/cm². Alternately, to be even more accurate and/or safer, the voltage drop at the actual electrodes could be measured by having a second high impedance electrode near the iontophoresis electrode(s). As a result, the total applied voltage could be increased until the point where the electrodes measure voltages associated with generation of electrolysis. At the point of electrolysis, the voltage increase could be halted, or even more desirably, could be slightly decreased to reduce electrolysis. Alternately, the pH at the actual electrodes could be measured with a pH sensitive electrode, and the total applied voltage could be increased until the electrodes begin significantly changing the local pH by electrolysis (at which point the voltage increase could be halted or the voltage decreased). In any or all of these cases, the current densities listed above are lower than the about 0.3 mA/cm² used by GlucoWatch, which leads us next to further background discussion on what current densities may be required and/or most desirable.

The current densities required for interstitial fluid extraction by reverse iontophoresis can also be compared to other ‘natural’ forms of iontophoresis in the body. A comparison and calculation is made here, with respect to the amount of natural iontophoresis that exists during sweat generation. These calculations are first-order and provide further background information only. Assume that at 1 nL/min/gland the eccrine sweat gland creates a secreted Na+ concentration of around 30 mM (the concentration in secretory coil is likely larger, because some amount of Na+ is reabsorbed by the duct, but such differences will be ignored for present purposes). Next, obtain the amount of Na+ in 1 nL: 1E-9 L*30E-3 Mol/L*6.02E23 Na+/Mol=1.8E13 Na+. Therefore, there is a Na+ generation rate of 1.8E13 Na+/min/gland. The flux of charged Na+ creates an equivalent electrical current (A, or C/s) of 1.8E13 Na+*1.6E-19 C/Na+=3 μC, which is 3 μC/min/gland. Turning that into C/s (for A), you obtain 0.05 μC/s/gland or 50 nA/gland. This Na+ current enters the secretory coil because there is a net negative charge induced (negative voltage) in the secretory coil caused by the injection of Cl− ions which are actively secreted by the cells lining the secretory coil. This Na+ current originates from interstitial fluid and enters the secretory coil through the tight junctions between the 1-2 layers of cells that line the secretory coil. This therefore represents a natural form of reverse-iontophoresis and therefore potentially a natural amount of electro-osmosis created in the secretory coil.

GlucoWatch generated 0.03 to 0.3 nL/min/gland of interstitial fluid using 300 μA/cm². For sweat, if we assume 100 active glands/cm², then the 50 nA/gland is equivalent to 5 μA/cm². Comparatively, GlucoWatch generates 0.03-0.3 nL/min/gland with 300 μA/cm², while sweat glands naturally generate 1 nL/min/gland with 5 μA/cm². Therefore, sweat has roughly 3-30× higher fluid flow rate, while using 60× less electrical current than GlucoWatch. This means that, at 1 nL/min/gland, if interstitial fluid were brought into the sweat by this natural form of reverse iontophoresis, the interstitial fluid component would be roughly 200-2000× less in volume than the sweat component. Because blood proteins are estimated to be 1000× or more dilute in sweat, this small interstitial fluid component suggests that even without electroporation, reverse iontophoresis could significantly increase the concentration of certain larger analytes in sweat.

Continuing the discussion from an applied/practical perspective, assume 0.1 nL/min/gland of sweat generation and the device applies only 5 μA/cm² of reverse iontophoresis current to the skin, then by first order calculation, and assuming the reverse iontophoresis (electro-osmosis) causes protein molecules enter sweat through the tight junctions or other pathways leading to the secretory coil, the device could receive 10× more protein than is found in normal sweat. This would be 60× less current than GlucoWatch. Or assume 0.1 nL/min/gland sweat generation rate, and apply only 50 μA/cm². The device could theoretically obtain 100× more protein with 6× less current than GlucoWatch. These examples suggest that reverse iontophoresis can enhance analyte concentrations at much lower current densities than those needed by GlucoWatch.

Returning to the previous discussion of current densities without electrolysis of water, about 0.1 mA/cm² to about 0.05 mA/cm² are well above the values of current densities shown above for increasing analyte concentrations in sweat. However, if the sweat generation rate were 1 nL/min/gland (not 0.1 nL/min/gland as just described above), then higher current densities would be needed to compensate for additional dilution of analytes in sweat. This reveals yet another need to reduce the sample volume for the sake of allowing sample collection at the lowest possible sample sweat rates, and as stated before, if there is no sweat, allowing interstitial fluid collection at the lowest possible current densities.

One more interesting comparison can be made. If only about 0.1 to 0.05 mA/cm² or less can be used if electrolysis is to be avoided, and if for GlucoWatch 0.3 mA/cm² was needed to generate 0.03 to 0.3 nL/min/gland of interstitial fluid, then trying to collect interstitial fluid only without electrolysis is certainly challenging if fast sampling intervals are needed. Clearly reduced volume between sensors and the skin surface is needed. Additionally, a combination of sweat and interstitial fluid can in some cases, allow a shorter sampling rate (faster transport of analytes) while requiring a lower current density.

Also in the prior art such as GlucoWatch, there is not a net flow of analyte to, and across, a sensor. Therefore, the chronological assurance or sampling interval is determined entirely by each time a sample is extracted (the frequency of sample extractions, is the chronological assurance and therefore determines the sampling interval). However, if a device were to have a net flow of analyte to, and across, a sensor, then the chronological assurance is not so simple as it is dependent on the sample volume required in the device and the sample generation and flow rates.

Lastly, in this background discussion of both challenges and opportunities presented by the disclosed invention, two more issues should be raised. First, electrolysis is not the only challenge caused by applying current through the skin. Skin damage, pain, discomfort, or annoyance in some cases could still be experienced by the user even at low current densities, and therefore reducing current densities nearly always improves user acceptance of such a device. Second, if a device transports sampled fluid away (actively, or passively), then electrical contact must still be adequately maintained with the skin, despite the reduced or absent fluid volume. Using a thick gel mitigates this issue completely, but results in a very large sample volume. Therefore, gels will need to be implemented with reduced volumes, electrodes must be kept in close contact with the skin, or the fluid volume between the device and skin must be minimized.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into intimate proximity with biofluid and analytes as they flow out from the skin surface. With such a new invention, non-invasive and wearable biosensing could become a compelling new paradigm as a biosensing platform.

### SUMMARY OF THE INVENTION

Embodiments of the disclosed invention provide biofluid sensing devices capable of reduced volume between the sensors and pre-existing pathways such as sweat glands, which decreases the sampling interval and/or reduces the required flow rate of the biofluid that is being generated. Some embodiments of the disclosed invention also mitigate challenges such as pH changes which can occur at an iontophoresis electrode.

In one embodiment, a sensor device for sensing on the skin includes one or more analyte-specific sensors and a volume-reducing component that provides a volume-reduced pathway for biofluid between the one or more sensors and pre-existing pathways in said skin when said device is positioned on said skin. In one embodiment, the biofluid may be more than 50% interstitial fluid. In another embodiment, the biofluid may be more than 50% sweat.

In other embodiments, various methods for integration of volume reducing components, sensors, chemical delivery components, and reverse iontophoresis components are provided. In yet another embodiment, various components and techniques are provided for buffering acid or base generation at an electrode for reverse iontophoresis.

### BRIEF DESCRIPTION OF THE DRAWINGS

The objects and advantages of the disclosed invention will be further appreciated in light of the following detailed descriptions and drawings in which:

FIG. 1A is a cross-sectional view of a wearable device for biosensing and reverse iontophoresis according to an embodiment of the disclosed invention.

FIG. 1B is a cross-sectional view of a wearable device for biosensing and reverse iontophoresis.

FIG. 1C is a cross-sectional view of a wearable device for biosensing and reverse iontophoresis.

FIG. 2 is a cross-sectional view of a wearable device for biosensing and reverse iontophoresis according to an embodiment of the disclosed invention.

FIG. 3 is a cross-sectional view of a wearable device for biosensing and reverse iontophoresis according to an embodiment of the disclosed invention.

FIG. 4 is a cross-sectional view of a wearable device for biosensing and reverse iontophoresis according to an embodiment of the disclosed invention.

FIG. 5A is a cross-sectional view of a wearable device for biosensing and reverse iontophoresis according to an embodiment of the disclosed invention.

FIG. 5B is a cross-sectional view of a wearable device for biosensing and reverse iontophoresis.

FIG. 6 is a cross-sectional view of a wearable device for biosensing and reverse iontophoresis according to an embodiment of the disclosed invention.

FIG. 7 is a cross-sectional view of a wearable device for biosensing and reverse iontophoresis according to an embodiment of the disclosed invention.

FIG. 8A is a cross-sectional view of a wearable device for biosensing and reverse iontophoresis according to an embodiment of the disclosed invention.

FIG. 8B is a cross-sectional view of a wearable device for biosensing and reverse iontophoresis according to an embodiment of the disclosed invention.

### DEFINITIONS

As used herein, “interstitial fluid” or “tissue fluid” is a solution that bathes and surrounds tissue cells. The interstitial fluid is found in the interstices—the spaces between cells (also known as the tissue spaces). Embodiments of the disclosed invention focus on interstitial fluid found in the skin and, particularly, interstitial fluid found in the dermis. In some cases where interstitial fluid is emerging from sweat ducts, the interstitial fluid contains some sweat as well, or alternately, sweat may contain some interstitial fluid. As used herein, “mainly interstitial fluid” means fluid that contains by volume less than 50% sweat (i.e., is primarily interstitial fluid). As used herein, “mainly sweat” means fluid that contains by volume 50% or greater of sweat (i.e., may contain some interstitial fluid, but has equal or greater amount of sweat than interstitial fluid). The percentages of each fluid can be quantified by several methods, such as measuring analyte dilutions in sweat (e.g., some analytes are dilute in sweat but not in interstitial fluid), or such as by measuring and comparing sample generation rates their respective contributions to the total fluid volume quantified (e.g., compare sample generation rates with or without application of reverse iontophoresis; or compare sample generation rates with or without natural or chemically-induced sweat stimulation).

As used herein, “biofluid” is a fluid that is comprised mainly of interstitial fluid or sweat as it emerges from the skin. For example, a fluid that is 45% interstitial fluid, 45% sweat, and 10% blood is a biofluid as used herein. For example, a fluid that is 20% interstitial fluid, 20% sweat, and 60% blood is not a biofluid as used herein. For example, a fluid that is 100% sweat or 100% interstitial fluid is a biofluid. A biofluid may be diluted with water or other solvents inside a device because the term biofluid refers to the state of the fluid as it emerges from the skin. Generally, as compared to blood, sweat is highly dilute of large sized analytes (e.g., greater than 1000× for proteins, etc.) and to a lesser extent, as compared to blood, interstitial fluid is dilute for some larger sized analytes (e.g., 10-100× or more or less depending on the specific analyte, current density, etc.).

As used herein, “pre-existing pathways” refer to pores, pathways, or routes through skin through which interstitial fluid may be extracted. Pre-existing pathways include but are not limited to: eccrine sweat ducts, other types of sweat ducts, hair follicles, inter-cell junctions, tape-stripping of the stratum corneum, skin defects, pathways created by electroporation of skin (e.g., of the stratum corneum), laser poration of skin, mechanical poration of skin (e.g., micro-needle rollers), chemical or solvent based poration of skin, or other methods or techniques. It should be recognized that “pre-existing” does not require that such pathways must be naturally occurring or that such pathways must exist prior to application of the device. Rather, methods of the disclosed invention may be practiced using a pathway that naturally exists or that was created for the particular application. Therefore, any technique to provide pre-existing pathways may be used in conjunction with embodiments of the disclosed invention. For example, a microneedle is a pre-existing pathway if the microneedle uses reverse iontophoresis for analyte extraction. However, generally, non-invasive access is preferred, and naturally occurring pre-existing pathways may be preferred for many applications. As another example, electroporation of the lining of the sweat glands may form or affect a pre-existing pathway. As another example, skin permeability enhancing agents or chemicals may form part or all of a pre-existing pathway. For simplicity of description herein, eccrine sweat glands will be the only pre-existing pathways explicitly discussed, but as noted above, embodiments of the disclosed invention may apply to any pre-existing pathway as defined above.

As used herein, “chronological assurance” means the sampling rate or sampling interval that assures measurement(s) of analytes in a biofluid in terms of the rate at which measurements can be made of new biofluid analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5-30 minute lag time between analytes in blood emerging in interstitial fluid), but the resulting sampling interval (defined below) is independent of lag time, and furthermore, this lag time is inside the body, and therefore, for chronological assurance as defined above and interpreted herein, this lag time does not apply.

As used herein, “interstitial fluid sampling rate” or “sweat sampling rate” or simply “sampling rate” is the effective rate at which new biofluid sample, originating from the pre-existing pathways, reaches a sensor that measures a property of the fluid or its solutes. Sampling rate is the rate at which new biofluid is refreshed at the one or more sensors and therefore old biofluid is removed as new fluid arrives. In an embodiment, this can be estimated based on volume, flow-rate, and time calculations, although it is recognized that some biofluid or solute mixing can occur. Sampling rate directly determines or is a contributing factor in determining the chronological assurance. Times and rates are inversely proportional (rates having at least partial units of 1/seconds), therefore a short or small time required to refill sample volume can also be said to have a fast or high sampling rate. The inverse of sampling rate (1/s) could also be interpreted as a “sampling interval” (s). Sampling rates or intervals are not necessarily regular, discrete, periodic, discontinuous, or subject to other limitations. Like chronological assurance, sampling rate may also include a determination of the effect of potential contamination with previously generated biofluid, previously generated solutes (analytes), other fluid, or other measurement contamination sources for the measurement(s). Sampling rate can also be in part determined from solute generation, transport, advective transport of fluid, diffusion transport of solutes, or other factors that will impact the rate at which new sample will reach a sensor and/or is altered by older sample or solutes or other contamination sources. During reverse iontophoretic extraction of fluid samples and analytes, some analytes that have a net charge could move faster or slower, with or against, the advective flow of fluid sample. In the event that the analytes are moving faster or slower than the advective flow, the sampling rate is still determined by the advective flow of interstitial fluid and the replenishment of new fluid sample across the sensor as the old sample is replaced. If an embodiment of the disclosed invention does not include a net flow of sample fluid across a sensor, and does include transport of a solute (analyte) to the sensor, then the term sampling rate may be replaced with the term “analyte sampling rate”. As will be described in greater detail below, sampling rate may be interpreted with respect to sensors that do not consume the analyte as part of the process of sensing the analyte, because these sensors are dependent on flow of fresh analyte to the sensors and removal of old analyte away from the sensors.

As used herein, “sweat stimulation” is the direct or indirect causing of sweat generation by any external stimulus. One example of sweat stimulation is the administration of a sweat stimulant such as pilocarpine or carbachol from a sweat stimulating component. Going for a jog, which stimulates sweat, is sweat stimulation, but would not be considered as sweat stimulating component. Sweat stimulation can include sudo-motor axon reflex sweating, passively diffused chemical into skin to stimulate sweat, or any other suitable method for sweat stimulation. As further examples, sweat stimulation can be achieved by simple thermal stimulation, by orally administering a drug, by intradermal injection of drugs such as methylcholine, carbachol, or pilocarpine, and by dermal introduction of such drugs using iontophoresis.

As used herein, “sample generation rate” is the rate at which biofluid is generated by flow through pre-existing pathways. Sample generation rate is typically measured by the flow rate from each pre-existing pathway in nL/min/pathway. In some cases, to obtain total sample flow rate, the sample generation rate is multiplied by the number of pathways from which the sample is being sampled. Similarly, as used herein, “analyte generation rate” is the rate at which solutes move from the body or other sources toward the sensors.

As used herein, “measured” can imply an exact or precise quantitative measurement and can include broader meanings such as, for example, measuring a relative amount of change of something. Measured can also imply a binary measurement, such as ‘yes’ or ‘no’ type qualitative measurements.

As used herein, “sample volume” is the fluidic volume in a space that can be defined multiple ways. Sample volume may be the volume that exists between a sensor and the point of generation of biofluid sample. Sample volume can include the volume that can be occupied by sample fluid between: the sampling site on the skin and a sensor on the skin where the sensor has no intervening layers, materials, or components between it and the skin; or the sampling site on the skin and a sensor on the skin where there are one or more layers, materials, or components between the sensor and the sampling site on the skin.

As used herein, “microfluidic components” are channels or other geometries formed in or by polymers, textiles, paper, or other components known in the art to transport fluid in a deterministic manner.

As used herein, “state void of sample” is where a space or material or surface that can be wetted, filled, or partially filled by a biofluid sample, but which is in a state where it is entirely or substantially (e.g., greater than 50%) dry or void of biofluid sample.

As used herein, “advective transport” is a transport mechanism of a substance or conserved property by a fluid due to the fluid's bulk motion.

As used herein, “diffusion” is the net movement of a substance from a region of high concentration to a region of low concentration. This is also referred to as the movement of a substance down a concentration gradient.

As used herein, a “volume-reduced pathway” or “reduced-volume pathway” is at least a portion of a sample volume that has been reduced by addition of a material, device, layer, or other component, which therefore increases the sampling interval for a given sample generation rate. A volume-reduced pathway can be created by at least one volume reducing component.

As used herein, “volume reducing component” means any component or material that reduces the sample volume and increases the sampling rate and/or the analyte sampling rate. In some cases, the volume reducing component is more than just a volume reducing material, because a volume reducing material by itself may not allow proper device function (e.g., the volume reducing material would need to be isolated from a sensor for which the volume reducing material could damage or degrade, and therefore the volume reducing component may comprise the volume reducing material and at least one additional material or layer to isolate volume reducing material from said sensors).

As used herein, “flux” is the rate of transfer of fluid and/or particles and/or solutes across a given surface. With respect to the eccrine sweat gland, flux can refer to both a fluid (e.g., interstitial fluid, intracellular fluid, etc.) and its contents, or refer to only one or more analytes entering into the sweat gland (e.g., ions, molecules, proteins, etc.). A flux in the sweat gland can occur at all areas, or in subsets of areas (e.g., a part of the dermal duct, or the secretory oil, etc.). A flux can also be referred to as “flux of analyte” or “analyte flux” or other similar uses that refer to a flux of analytes in interstitial fluid, moving along with or against the flow of one or more of these fluids, or moving fully or somewhat independently of flow of these fluids. For example, charges of analytes can be negative or positive, and fluxes can be in the opposite direction of advective flow.

As used herein, “reverse iontophoresis” is a subset or more specific form of “iontophoresis” and is a technique by which electrical current and electrical field cause molecules to be removed from within the body by electroosmosis and/or iontophoresis. Although the description below focuses primarily on electro-osmosis, the term “reverse iontophoresis” as used herein may also apply to flux of analytes brought to or into the devices of the disclosed invention, where the flux is in whole or at least in part due to iontophoresis (e.g., some negatively charged analytes may be transported against the direction of electroosmotic flow and eventually onto a device according to an embodiment of the disclosed invention). Electroosmotic flow (or electro-osmotic flow, synonymous with electroosmosis or electroendosmosis) is the motion of liquid induced by an applied potential across a porous material, capillary tube, membrane, microchannel, or any other fluid conduit. Because electroosmotic velocities are independent of conduit size, as long as the electrical double layer is much smaller than the characteristic length scale of the channel, electroosmotic flow is most significant when in small channels. In biological tissues, the negative surface charge of plasma membranes causes accumulation of positively charged ions such as sodium. Accordingly, fluid flow due to reverse iontophoresis in the skin is typically in the direction of where a negative voltage is applied (i.e., the advective flow of fluid is in the direction of the applied electric field). As used herein, the term “iontophoresis” may be substituted for “reverse iontophoresis” in any embodiment where there is a net advective transport of biofluid to the surface of the skin. For example, if a flow of sweat exists, then negatively charged analytes may be brought into the advectively flowing sweat by iontophoresis. The net advective flow of sweat would typically be needed, because in this case, a net electro-osmotic fluid flow would be in the direction of sweat into interstitial fluid (and without a net advective flow of sweat, the sweat would be lost, and there would be no pathway for transporting the analyte to at least one sensor). Furthermore, because “reverse iontophoresis” is a subset or more specific form of “iontophoresis”, the term “iontophoresis” may refer to both “reverse iontophoresis” and “iontophoresis”. The terms “reverse iontophoresis” and “iontophoresis” are interchangeable in the disclosed invention.

As used herein, the term “analyte-specific sensor” is a sensor specific to an analyte and performs specific chemical recognition of the analytes presence or concentration (e.g., ion-selective electrodes, enzymatic sensors, electrically based aptamer sensors, etc.). For example, sensors that sense impedance or conductance of a fluid, such as biofluid, are excluded from the definition of “analyte-specific sensor” because sensing impedance or conductance merges measurements of all ions in biofluid (i.e., the sensor is not chemically selective; it provides an indirect measurement). Sensors could also be optical, mechanical, or use other physical/chemical methods which are specific to a single analyte. Further, multiple sensors can each be specific to one of multiple analytes.

As used herein, the term “sensor that consumes the analyte” is an analyte-specific sensor that decreases the total amount of analyte present (e.g., glucose and other enzymatic/amperometric sensing).

As used herein, the term “sensor that does not consume the analyte” is an analyte-specific sensor that responds by equilibrating to the local concentration of the analyte (e.g., ionselective or electrochemical aptamer-based sensors) and that does not decrease the total amount of the analyte present. An aptamer-based sensor may bind an analyte, but the analyte is not consumed (i.e., once the analyte binds, the same site will not bind further analyte, and furthermore, the analyte can be released back into solution as well). The definition and calculations for sampling rate and sampling interval described herein apply to cases where the sensors do not consume the analyte.

As used herein, the terms “wicking pressure,” “wicking force,” “capillary pressure,” or “capillary force” means a pressure or force that should be interpreted according to its general scientific meaning. For example, capillary (tube) geometry can be said to have a capillary pressure or a wicking pressure. For example, a wicking textile or gel may have a capillary pressure, even if the material is not geometrically a tube or a channel. Similarly, the (relatively empty) space between a material placed on skin and the skin surface can have an effective wicking pressure. The terms wicking or capillary pressure and wicking or capillary force may be used interchangeably herein to describe the effective pressure provided by any component or material that is capable of capturing biofluid by a negative pressure (i.e., pulling it into or along said component or material). For simplicity, the term “wicking pressure” is used herein to refer to any of the above alternate terms. Wicking pressure also must be considered in its specific context, for example, if a sponge is fully saturated with water, then it has no remaining wicking pressure. Therefore, wicking pressure as used herein describes a device and/or a component during use, and not interpreted in isolation or in contexts other than the disclosed devices or use scenarios.

As used herein, the term “wicking collector” means a component of the disclosed invention that supports the creation of, or sustains, a volume reduced pathway by use of wicking pressure, and/or that is the wicking element adjacent to or on skin that receives biofluid before it reaches a sensor. A wicking collector can be a microfluidic component, a capillary material, a wrinkled surface, a textile, a gel, a coating, a film, or any other suitable component. A single component may serve multiple functions as a wicking collector and, for example, a wicking pump (defined below).

As used herein, the term “wicking pump” refers to a component that supports creation of or sustains a volume reduced pathway by use of wicking pressure, or that receives biofluid after a sensor and has a primary purpose of collecting excess biofluid to allow sustained operation of the device. A wicking pump may also include an evaporative material or surface that is configured to remove excess biofluid by evaporation of water. A wicking pump may be part of the same component or material that serves other purposes (e.g., a wicking collector or a wicking coupler), and in such cases, the portion of said component or material that at least in part receives biofluid after the sensor(s), is also a wicking pump as defined herein.

The term “wicking pump” may also reference alternate configurations, such as a small mechanical pump, or osmotic pressure across a membrane (i.e., the wicking pump would be the membrane and the draw solution or material), so long as the pressure generated satisfies the requirements described herein, and the other materials or components between the wicking pump and skin operate by wicking pressure to maintain their respective sample volumes.

As used herein, the term “wicking coupler” refers to a component that is on or adjacent to a biofluid sensor and that promotes the transport of biofluid or its solutes (e.g., by advective flow, diffusion, or other method of transport) between another wicking component or material and a sensor. In some embodiments, a single component may function as both a wicking coupler and a wicking collector. In other embodiments, a sensor may be configured with a wicking surface or material that functions without a wicking coupler (e.g., an immobilized aptamer layer which is hydrophilic, or polymer ionophore layer which is porous to the analyte). A wicking coupler may be part of the same component or material that serves other purposes (e.g., a wicking collector or a wicking pump), and in such cases, the portion of said component or material that, at least in part, couples biofluid to a sensor(s) and that is on or adjacent to the sensor(s), is also a wicking coupler as defined herein.

As used herein, the term “wicking space” refers to the space between the skin and wicking collector that would be filled by air, skin oil, or other non-biofluid fluids or gases if no biofluid existed. In some embodiments of the disclosed invention, even if biofluid exists, the wicking collector removes some or most of biofluid from the wicking space by action of wicking pressure provided by the wicking collector.

As used herein, “biofluid collector pressed against skin” is a component that at least in part is pressed directly against the skin, and which is at least a part of a volume-reducing component. Further, a biofluid collector includes a plurality of pores or pathways in a material and/or on the surface of a material that is held against skin so that the plasticity of skin allows skin defects, hair, and other sample volume increasing aspects of skin to at least partially conform against the material.

As used herein, “space between skin and a biofluid collector pressed against skin” refers to the space between the skin and a biofluid collector pressed against skin that would be filled by air, skin oil, or other non-sweat fluids or gases if no sweat existed.

As used herein, “pressure element” is any component that at least in part provides pressure to a biofluid collector pressed against skin to create at least in part a reduced sample volume in the space between skin and a biofluid collector pressed against skin.

### DETAILED DESCRIPTION OF THE INVENTION

Embodiments of the disclosed invention apply at least to any type of sensor device that measures at least one analyte in interstitial fluid extracted at least in part by reverse iontophoresis through pre-existing pathways. Further, embodiments of the disclosed invention apply to sensing devices which measure chronological assurance. Further, embodiments of the disclosed invention apply to sensing devices which can take on forms including patches, bands, straps, portions of clothing, wearables, or any suitable mechanism that reliably brings sampling and sensing technology into intimate proximity with biofluid sample as it is transported to the skin surface. While some embodiments of the disclosed invention utilize adhesives to hold the device near the skin, devices could also be held by other mechanisms that hold the device secure against the skin, such as a strap or embedding in a helmet. Certain embodiments of the disclosed invention show sensors as simple individual elements. It is understood that many sensors require two or more electrodes, reference electrodes, or additional supporting technology or features which are not captured in the description herein. Sensors are preferably electrical in nature, but may also include optical, chemical, mechanical, or other known biosensing mechanisms. Sensors can be in duplicate, triplicate, or more, to provide improved data and readings. Certain embodiments of the disclosed invention show sub-components of what would be sensing devices with more sub-components needed for use of the device in various applications, which are obvious (such as a battery), and for purposes of brevity and of greater focus on inventive aspects, such components are not explicitly shown in the diagrams or described in the embodiments of the disclosed invention.

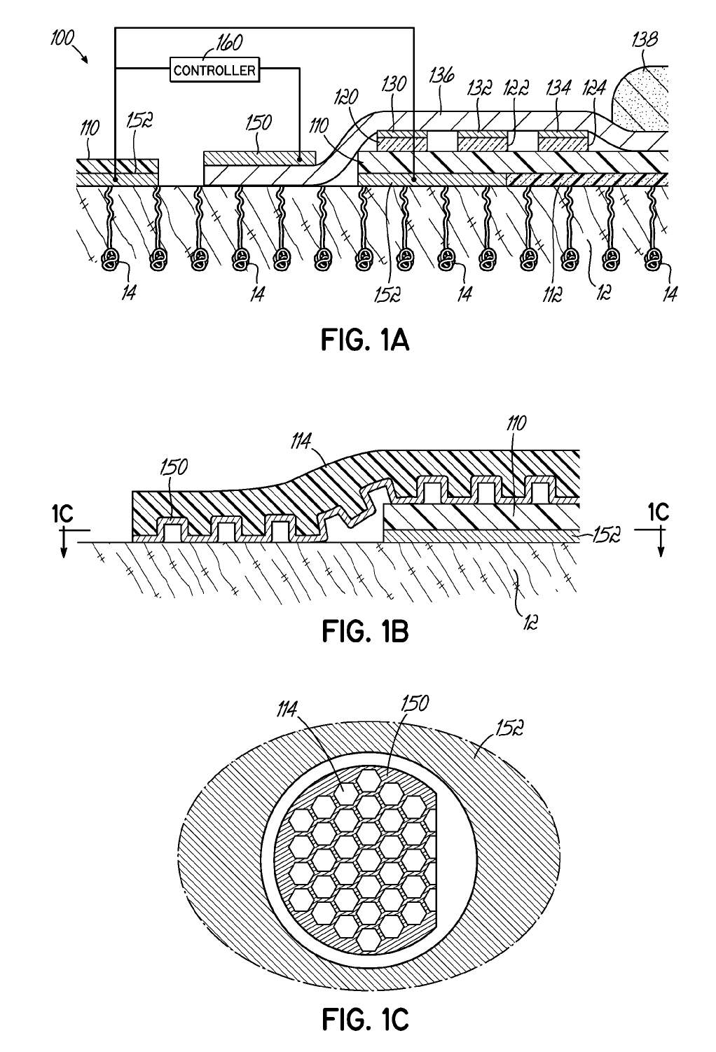

With reference to FIG. 1A, in an embodiment of the disclosed invention, a portion of a device 100 is shown positioned on the skin 12, which contains pre-existing pathways such as sweat glands 14. The device 100 may be configured or implemented to work with a biofluid such as interstitial fluid or sweat to provide a reduced sample volume by creating a volume-reduced pathway with a volume-reducing component. The device 100 includes polymer substrates 110, such as PET, and a skin adhesive 112, such as those commercially sold by 3M Corporation. The polymer substrates 110 may serve a variety of functions such as the physical support of one or more elements of the device 100 or such as water impermeability. The device 100 further includes one or more analyte-specific sensors 120, 122, 124, at least one of which is a sensor that does not consume its target analyte. The device 100 is capable of applying reverse iontophoresis to generate a flow of sweat or interstitial fluid and includes an electrically conductive metal and gel counter electrode 152, like those used commercially for iontophoresis or skin electrical monitoring, and a reverse iontophoresis electrode 150 for bringing biofluid and/or one or more desired analytes, into a pre-existing pathway. In cases where there is not an advective flow of sweat, the reverse iontophoresis electrode 150 may also bring interstitial fluid into the device 100. The device 100 further includes wicking couplers 130, 132, 134 positioned between a wicking collector 136 and the sensors 120, 122, 124. To remove older or excess biofluid, a wicking pump 138 is fluidically coupled to the wicking collector 136.

With reference to FIGS. 1B and 1C, in an embodiment, an exemplary configuration of the wicking collector 136 and the counter electrode 152 of the device 100 is shown. The wicking collector 136 is illustrated as a microreplicated polymer 114, such as PET. The microreplicated polymer 114 contains a network or grid of biofluid wicking channels or pathways that collects biofluid from the skin 12 and transports it to at least one of the sensors 120, 122, 124 (not shown in FIG. 1B). In another embodiment, a wicking collector includes a network of wicking pathways formed by, for example, hot-stamping a network of agar hydrogel channels on a planar PET surface, which could have similar wicking properties and geometries as the physical channels in the microreplicated polymer 114. The electrode 150 provides a current for reverse iontophoresis and could be constructed of, for example, hydrophilic gold, agar hydrogel coated carbon electrodes, or other suitable materials including buffered materials described herein. In an embodiment where the electrode 150 is consumed during buffering (such as Ag, Ag/Cl), the elements 114 and 150 may be a single electrode material that is buffering. Preferably, the network of wicking pathways in the wicking collector 136 comprises less than 50% of the available horizontal surface area so that the effective sweat volume of the device 100 is reduced by a factor of 2× compared to a continuous planar sheet of wicking material. Further, the wicking collector 136 may comprise less than 30%, less than 20%, or less than 10% of the available surface area where the wicking collector 136 is adjacent to the skin 12, which would reduce the effective sample volume by roughly 3×, 5×, or 10×, respectively. Such reduced surface area will be taught in an example below. Although a hexagonal network is shown, any suitable network is possible (e.g., linear, square, irregular, tree root pattern, etc.).

With further reference to FIGS. 1A and 1B, in an embodiment of the disclosed invention, the electrode 150 is electrically grounded with respect to the device 100 and any one of the sensors 120, 122, 124, and the voltage for reverse iontophoresis is applied by the counter electrode 152. In other words, the reverse iontophoresis electrode 150 may be at the same electrical potential of at least one of the analyte-specific sensors 120, 122, 124 during the process of reverse iontophoresis. Voltages that are relatively close in magnitude, such as within several hundred mV, can be considered to be at the same electrical potential. As a result, the sensors 120, 122, 124 do not experience the reverse iontophoresis voltage that could interfere with or damage such sensors.

With reference to FIG. 1A, the wicking collector 136 has a greater wicking pressure than the pressure of the wicking space between the skin 12 and the wicking collector 136. Therefore, as biofluid emerges onto the skin 12, it will contact the wicking collector 136 forming a sample volume that excludes a portion of the wicking space. Materials capable of providing adequate wicking pressure are well-known by those skilled in the art. Further, those skilled in the art can alter a material's wicking pressure to a desired level through, for example, control of capillary geometry or surface energy control. If the wicking pressure were strong enough to void the wicking space of all biofluid, then a poor electrical connection with pre-existing pathways for reverse iontophoresis could exist. During sweating, such voiding of the wicking space may not be an issue, as sweat would reestablish the electrical connection as it emerges from sweat ducts 14 and contacts the wicking collector 136. However, with no sweating, embodiments that include, for example, a network of wicking pathways made of agar hydrogel may be preferred as they always remain wetted with biofluid and can better maintain electrical contact with the pre-existing pathways. Furthermore, a device applied with pressure may similarly reach a conformal state with skin that ensures proper electrical contact. Therefore, an embodiment of the disclosed invention may include a wicking collector that is electrically conductive, and/or may include a wicking collector that maintains electrical contact with pre-existing pathways.

With further reference to FIG. 1A, the wicking collector 136 has a wicking pressure greater than or equal to that of wicking pump 138 to ensure adequate wicking pressure by the wicking collector 136 and to maintain sufficient biofluid sample contact with the sensors 120, 122, 124. In the presence of biofluid, the wicking collector 136 will tend to become saturated, at which point its wicking pressure would approach zero, and its ability to provide a reduced sample volume would be compromised. Therefore, biofluid must be continuously removed to prevent wicking collector 136 from becoming saturated. To remove excess biofluid, the device 100 may be configured with a wicking pump 138 that is in fluid communication with the wicking collector 136. In order to ensure adequate wicking pressure by wicking collector 136 and to maintain sufficient biofluid sample contact with sensors 120, 122, 124, the wicking collector 136 may have wicking pressure greater than or equal to that of wicking pump 138. In addition, the wicking pump 138 must have sufficient volume (i.e., fluidic capacity) to sustain operation of the device 100 throughout the application's intended duration (i.e., it must not become saturated during device operation). For example, if the device is to be used for 24 hours, then the wicking pump 138 should not become fully saturated with biofluid during the 24 hours of operation. In some embodiments, wicking collector 136 and wicking pump 138 may be the same material or component.

Still referring to FIG. 1A, the sample volume of the wicking collector 136 may be less than the sample volume of the wicking space between the wicking collector 136 and skin 12. Otherwise, adding a wicking collector 136 would primarily increase the sample volume, which would tend to increase the sampling interval. This may be accomplished by varying the thickness, area, or porosity of the wicking collector 136. Textiles, paper or other common wicking materials will often fail this criteria, since they are typically more than 100 μm thick, although these materials are not precluded from forming a wicking collector. In an embodiment where at least a portion of the area of a wicking collector does not interface with, or is not adjacent to, skin (e.g., wicking collector 136), only the portion of the wicking collector that interfaces with or is adjacent to skin should have a sample volume that is less than that of wicking space between the wicking collector and the skin. An embodiment may have, for example, a wicking space between the wicking collector 136 and the skin 12 with an average height of 50 μm due to skin roughness (more if hair or debris is present), and the wicking collector 136 may be comprised of a 5 μm thick layer of screen printed and hydrophilic nano-cellulose. In this embodiment, the sample volume would be reduced by roughly 10× compared to a similar device with no wicking collector. Other methods and materials may be used to form a suitable wicking collector. Where a device is applied loosely to skin, a low-volume wicking collector may not be important. However, in such cases, the sample volume would already be impractically large.

With further reference to FIG. 1A, the design of the wicking couplers 130, 132, 134, wicking collector 136, and wicking pump 138 may vary. Some embodiments may include a wicking coupler 130, 132, 134. In one embodiment, the wicking coupler will have greater than or equal wicking pressure than the all the other wicking components. This will ensure that sensors remain wetted with biofluid. Because the wicking couplers 130, 132, 134 are porous to biofluid, new biofluid may replace old biofluid by advective flow, or without advective flow (by primarily diffusion). Wicking couplers 130, 132, 134 that are adequately thin (e.g., 10's of μm or less) can allow rapid diffusion of analytes to and from the biofluid to the sensors 120, 122, 124, which is equivalent to replacing old biofluid with new biofluid.

The wicking space can change over time due to skin plasticity, for example, skin can swell and become smoother as it hydrates, and skin can flatten if a device applies pressure against the skin surface. Therefore, in an embodiment of the disclosed invention, if the sample volume is to be reduced between the skin 12 and the area of the wicking collector 136 on or adjacent to skin, then at the time of first application of the device 100 to skin, the sample volume of the portion of the wicking collector 136 that interfaces with or is adjacent to skin is less than the sample volume of the wicking space between the wicking collector 136 and skin 12.

In an embodiment, a wicking collector could be constructed of Rayon or other material that has two or more levels of wicking pressures. For example, Rayon has a first and greater wicking pressure when fluid is wicked along grooves in its fibers, and a second and lower wicking pressure when fluid also fills the spaces in between such fibers. Alternately, open-faced rectangular micro-channels could have a higher wicking pressure when they have less biofluid in the channels (i.e., when only wicking along the corners of the channels which have the highest wicking pressure instead of filling the channels). Therefore, an embodiment of the disclosed invention may include a wicking material where the sample volume in said wicking material during use is less than 50% of the total available volume of such said wicking material.

In use, the device 100 may be placed on a person's skin to sense a biofluid. The following exemplary use of the device 100 is described relative to interstitial fluid, although the description applies equally to any biofluid as defined above. The skin adhesive 112 secures the device 100 to the skin 12. The reverse iontophoresis electrode 150 and the counter electrode 152 are used to generate a flow of interstitial fluid. The wicking collector 136 transports interstitial fluid from the skin 12 towards the wicking pump 138. As interstitial fluid moves through the wicking collector 136, the wicking couplers 130, 132, 134 allow the sensors 120, 122, 124, respectively, to sense the interstitial fluid. In an exemplary embodiment, the sensor 120 may comprise an ion-selective electrode for sodium and a reference electrode, the sensor 122 is an amperometric sensor for urea, and the sensor 124 is an electrochemical aptamer sensor for vasopressin.

In an aspect of the disclosed invention, a net advective flow of biofluid from the skin to the sensor(s) in the device is required for the sensor(s) to sense the desired analytes in the biofluid. As previously noted, the terms “iontophoresis” may be substituted for “reverse iontophoresis” in any embodiment for cases where sweat is the primary driver of a net advective transport of biofluid to the surface of the skin. If a flow of sweat exists, then negatively charged analytes, such as acidic analytes or certain proteins or peptides, may be brought into the advectively flowing sweat by iontophoresis. The net advective flow of sweat is a requirement if “iontophoresis” is to be substituted for “reverse iontophoresis” because, in this case, a net electro-osmotic fluid flow would be in the direction of sweat into interstitial fluid (and without a net advective flow of sweat, there would be no pathway for transporting the analyte to at least one sensor as illustrated in embodiments of the disclosed invention). Even if there were a fluid pathway for pure iontophoretic transport of an analyte (i.e., no advective flow) to a sensor, iontophoretic currents are typically dominated by small ions such as Cl⁻, and few of the other possible negatively charged analytes could be brought to a sensor in meaningful quantities.

With further reference to FIG. 1A, analytes may be diluted in biofluid and the dilution degree may also be unpredictable. Therefore, in an embodiment of the disclosed invention, ratios of two or more analytes can be measured by two or more respective analyte specific sensors. For example, a first sensor (e.g., sensor 122) could measure a first cytokine and a second sensor (e.g., sensor 124) could measure a second cytokine that has similar dilution in the biofluid as the first cytokine (e.g., due to size, charge, lipophilicity, etc.), and the ratios of these two analytes may be compared at one time point or over multiple time points to provide meaningful information (e.g., by the controller 160). For example, ratios of measurements of Cortisol and DHEA may be compared over time, or ratios between a pro-inflammatory and an anti-inflammatory cytokine may be compared. Another embodiment of the disclosed invention may include at least a first analyte specific sensor for a first analyte and at least a second analyte specific sensor for a second analyte where said first analyte and said second analyte have similar expected dilutions in the biofluid. For example, analytes having a similar dilution in biofluid may be two hydrophilic analytes each with a molecular weight of about 1000 Da, or two proteins that each has a molecular weight of greater than 20 kDa.

With further reference to FIG. 1A, in an embodiment of the disclosed invention, the reverse iontophoresis electrode 150 may be used as needed to cause generation of interstitial fluid through pre-existing pathways with or without the presence of sweat secretion from the sweat ducts 14. The wicking collector 136 is also electrically conductive between reverse iontophoresis electrode 150 and pre-existing pathways. To that end, in various embodiments, the wicking collector 136 may at least partially comprise a conductive fluid (e.g., a hydrogel or textile filled with biofluid, etc.) or may be a porous membrane, textile, or microfluidic component that has been plated with a hydrophilic and electrically conductive coating of, for example, gold.

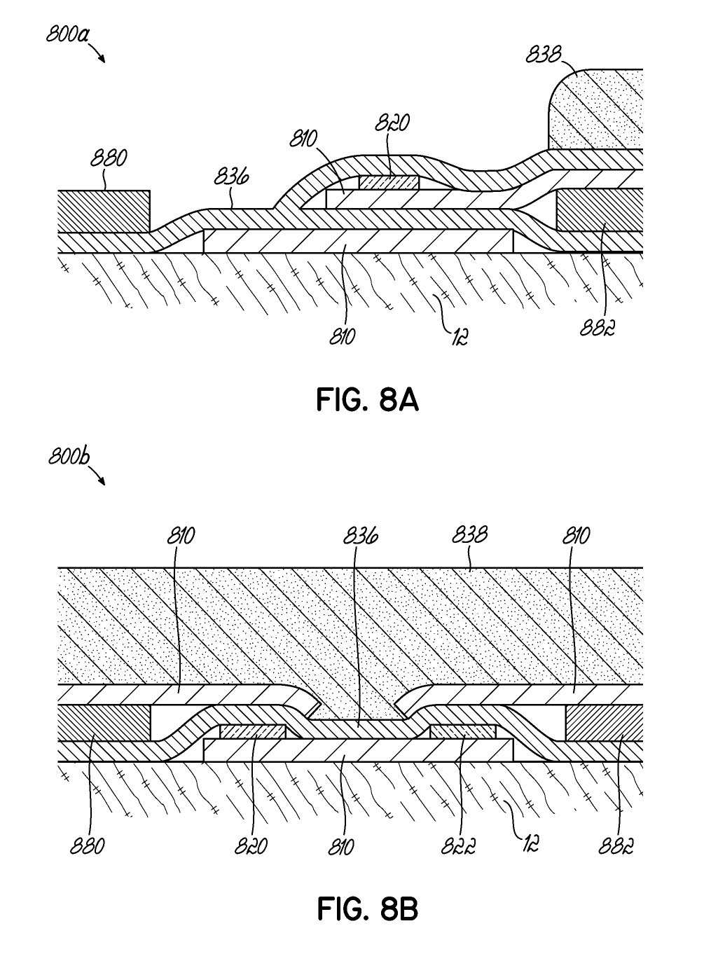

With further reference to FIG. 1A, an illustrative use of the device 100 includes the use of periodic reverse iontophoresis for monitoring dehydration. In such an application, it may be desirable to track water loss through measuring sweat generation rate at an unstimulated sweat sensing site (e.g., Na+ concentration, skin impedance, and/or a flow meter) and also through measuring a dehydration biomarker (e.g., vasopressin) every hour at a site where interstitial fluid is extracted by reverse iontophoresis. Alternately, in another embodiment, both the sweat and interstitial fluid sampling could be at the same site where sweat stimulation and reverse iontophoresis would be applied as necessary. Vasopressin might be the only analyte sampled by reverse iontophoresis (e.g., if vasopressin could not be measured in sweat because of dilution/filtration due to its relative large molecular weight). Urea could be measured in the sweat sample or the interstitial fluid sample and be used to help determine the hydration state. Therefore, an embodiment of the disclosed invention may also include at least two analyte-specific sensors with at least one for an analyte in sweat and at least one for an analyte in interstitial fluid. These concepts will be discussed in additional detail for FIG. 8. If the measurements of vasopressin were hourly, then there would be no need to continuously perform reverse iontophoresis to obtain vasopressin. For example, the reverse iontophoresis could be applied for less than 15, 6, or 3 minutes each hour, which would be less than 25%, 10%, or 5% of the total time of use compared to continuous reverse iontophoresis. As a result, the total amount of reverse iontophoresis is dramatically reduced. Once a volume reduced pathway is established, for example by constant sweating, it may be that no warm-up period is required before applying reverse iontophoresis immediately, which could allow access to the analyte in the interstitial fluid in mere minutes. Therefore, an embodiment of the disclosed invention may include sensing of at least one analyte in sweat with no warm-up period for the analyte sampling.

With further reference to FIG. 1A, an illustrative use of the device 100 includes the use of on-demand reverse iontophoresis for measuring the luteinizing hormone for fertility monitoring. The device 100 may include a controller 160, which may act as an activation component for iontophoresis and which could be part of the electronics. For continuous iontophoresis (i.e., not on demand), a simple current or voltage source may be suitable. For prolonged monitoring, a new device or a new disposable portion of a device could be applied each day. On demand reverse iontophoresis could be initiated at a set time each day, at an opportune time determined by the user. For example, in an embodiment, the reverse iontophoresis could be initiated by the user, because the user would determine that it is an opportune time to measure for luteinizing hormone if the user was intending to become pregnant. As a result, in some cases, reverse iontophoresis for a user may only occur once or very few times per month. Additionally, the reverse iontophoresis may be initiated based at least in part on feedback from the device 100. For example, the device 100 may measure estrogen and progesterone in sweat or some other biomarker, such as Cl− concentration to determine the body's thermal set-point indication, any or all of these provide an indication that ovulation is approaching or has occurred. Once the sensors provide measurements of sweat estrogen and/or progesterone, or another analyte such as Cl−, that indicate luteinizing hormone detection is more likely, the activation component may initiate reverse iontophoresis. In other words, an activation component for iontophoresis may be in electronic communication with an analyte-specific sensor to determine when to initiate reverse iontophoresis. Reverse iontophoresis may also be initiated a plurality of times following the original initiation to ensure an accurate reading.

With further reference to FIG. 1A, an illustrative use of the device 100 includes applying reverse iontophoresis with pulses or a frequency that is adequate to bring interstitial fluid into the dermal duct, but not substantially into the secretory coil of sweat glands 14. The potential advantage of this approach is that it may avoid iontophoretic interference with sweat production by the eccrine gland (e.g., reverse iontophoresis is well-known to be used to treat hyperhidrosis). For example, by treating plasma membranes as electrical capacitors and taking into consideration the conductivity of sweat in the gland and the dimension of the sweat duct, an example RC time constant for the dermal duct can be calculated to be around 1 to 10 ms. Therefore, if reverse iontophoresis is applied with high frequency waveforms or pulses and with voltage oscillation times of 1-10 ms or less, the voltage may not penetrate to the secretory coil of sweat glands 14. Therefore, in an embodiment, the application of reverse iontophoresis may include a plurality of waveforms with individual durations of less than 10 ms each.

With further reference to FIG. 1A, prolonged or repeated reverse iontophoresis may result in changes to the pH of the biofluid at or near the electrodes 150, 152. Accordingly, in an embodiment, the device applies reverse iontophoresis with short duration pulses that do not significantly alter the biofluid pH. For example, assume a 10 μm thick wicking collector 136, which defines a sample volume of 10E-4 cm*1 cm²=1 μL/cm² between the reverse iontophoresis electrode 150 and the skin 12. Using first principles calculations, at 1 nL/min/gland of sweat generation rate and 100 glands/cm² (100 nL/min/cm²), this 10 μm wicking collector 136 would refill with new biofluid roughly every 10 minutes. If the skin was exposed to continuous reverse iontophoresis at 5 μA/cm², then the pH of the biofluid would change from near 7 to 1.5 (i.e., between the pH of lemon juice and stomach acid) and pH of 12.5 under the electrodes depending on polarity. Therefore, an embodiment of the disclosed invention includes a ratio of current per area (A/cm²) to biofluid generation rate (L/min/cm²) that is less than 50 A/L/min (i.e., 5E-6 A/100E-9 L/min).

With further reference to FIG. 1A, in an embodiment of the disclosed invention, acid or base accumulation is remedied by periodically reversing the polarity of the electrodes 150 and 152. For example, with the reverse iontophoresis electrode 150 electrically grounded, the counter electrode 152 could have a positive voltage for 5 minutes. Next, a 25 minute rest period of no voltage could occur. Next, for 5 minutes, the voltage polarities could be applied in reverse fashion, reversing acidic accumulation to be more basic, and vice-versa. Next, a 25 minute rest period of no voltage could occur. As a result, reverse iontophoresis is applied and the effects of pH changes are further mitigated or eliminated, and hourly readings with increased analyte fluxes are provided.

With further reference to FIG. 1A, in an embodiment of the disclosed invention, because changes in pH can alter the readings at a sensor, sensors 120, 122, 124 may include a pH sensor to correct for pH induced changes in an analyte-specific sensor. For reverse iontophoresis, the times, durations, magnitudes, and other parameters related to pH or other possible confounding factors are heavily dependent on the electrode areas, spacing, connection to the skin, sample generation rates, and other factors. The pH sensor(s) could be used to safely provide feedback control by allowing reverse iontophoresis current density or duration to be increased until a pH limit is reached, as measured by a pH sensor. In other words, a pH sensor may be used to determine limits for the amount of reverse iontophoresis that is applied.

With further reference to FIG. 1A, in an embodiment of the disclosed invention, either or both of the electrodes 150, 152 could be at least partially constructed of or coated with a buffering material. Exemplary materials include silver and silver chloride, which increase buffering of pH. Oxidation results in formation to insoluble silver chloride at the anode, consuming chloride ions from solution. With a silver chloride-coated silver cathode (e.g., a wire, a plate, etc.), the current reduces the silver chloride to silver, releasing the chloride ion. Other exemplary buffering materials include polymers that incorporate buffering groups such as COO⁻, NH₃⁺, or other suitable buffering groups, chemicals such as acids or bases, or commercial buffers such as TAPS, Bicine, Tris, Tricine, TAPSO, HEPES, TES, MOPS, PIPES, Cacodylate, or MES. It should be recognized that components other than a sensor may include one or more buffering agents for regulating pH.

With further reference to FIG. 1A, in an embodiment of the disclosed invention, the electrode sizes for electrodes 150, 152 could be designed to mitigate issues with pH caused by electrical current passing into the skin 12. For example, the reverse iontophoresis electrode 150 could be buffered using one or more approaches as described herein, and the counter electrode 152 may not be buffered but may have a larger area by at least 2×, 10×, or 20×. In that manner, the counter electrode has a lower current density and, therefore, a reduced pH build up. Further, the electrode area and skin electrical contact area need not be equal to each other. For example, the electrode 150 could be 0.2 cm² in area; the electrode 150 is in contact with the wicking collector 136, which is electrically conductive because it is filled with sweat, that has an electrical contact area with skin that is less than 0.1 cm². As a result, a threshold current/density at the skin 12 for extracting interstitial fluid density could be reached with a current density at the electrode 150 that is at least half of the current density at the skin 12. As a result, changes in pH could be reduced. Therefore, an embodiment of the disclosed invention may include at least one reverse iontophoresis electrode with at least 2× greater area than the area of electrical contact with the skin.

In an aspect of the disclosed invention, the electrode sizes may be designed to mitigate issues with pain or discomfort caused by electrical current passing into the skin 12. Pain or discomfort caused by electrical current in skin does not scale linearly in terms of the relationship of current density to electrode area as taught in P. W. Ledger, Skin biological issues in electrically enhanced transdermal delivery (1992). The smaller the electrode area is, generally the larger the current density that can be used without a perception of the current or perception of pain. For example, an electrode of 24 cm² area generates a tingle at 0.08 mA/cm², whereas an electrode of 0.64 cm² generates a tingle at 0.4 mA/cm² (varies based on location on skin and from person to person). In an aspect of the disclosed invention, due to the reduced sample volumes, the areas of electrical contact with skin for reverse iontophoresis are reduced. Consider for example, sampling biofluid from pre-existing pathways that are sweat ducts with densities of 100 glands/cm², then the contact areas needed to cover an average of 5, 10, and 50 glands would be 0.05 cm², 0.1 cm², and 0.5 cm², respectively. With sweat ducts with densities of 200 glands/cm², then the contact areas needed to cover an average of 5, 10, and 50 glands would be 0.025 cm², 0.05 cm², and 0.25 cm², respectively. Even fewer glands could be covered, so the above areas of contact may represent upper limits for contact areas for one or more embodiments of the disclosed invention. These areas can be of the electrodes themselves or, in the case of intervening materials or layers between the electrodes and skin, can represent the electrical contact area with skin.

In an aspect of the disclosed invention, sample volumes are dramatically reduced compared to the prior devices. However, reduced sample volumes can also cause issues with analyte depletion in the biofluid (e.g., the sensor captures analytes and thereby changes analyte concentration in biofluid, causing the sensor to erroneously measure analyte concentration). For example, consider a sensor with an area of about 0.001 cm² (about 300 μm×300 μm) with 5E12 aptamer probes/cm², which is 5E9 probes or about 8E-15 moles of probe. Now, assume for 14.1 nL of solution that flows past the sensors includes 100 nM of cortisol. That is 14.1E-9 L*100 nM/L=1.41E-15 moles of cortisol. There are about 6× fewer available analytes than available probes. Therefore, a sensor with an area of about 0.001 mm² (about 30 μm×30 μm) might be preferred because it would contain about 8E-17 moles of probe, which is much less than the moles of analyte and therefore the analyte will not be depleted in the sample. For higher concentration analytes (e.g., 100 μM), preferred sensor areas might therefore be about 1 mm². Furthermore, because embodiments of the disclosed invention work with such small sample volumes, smaller sensor areas are preferred because a larger sensor area would increase the sample volume required for the sensor. Exemplary sensor areas include less than 0.001 mm², less than 0.01 mm², less than 0.1 mm², or less than 1 mm².

In another aspect of the disclosed invention, the entry of solutes into the secretory coil or sweat duct can be enhanced using non-natural (applied) reverse iontophoresis. As previously described, Na+ enters the secretory coil from the interstitial fluid through the cell-cell junctions or “tight junctions” between cells. When a flux of Na+ is driven by an electric field; the moving Na+ (and other positive ions) drags additional interstitial fluid and possibly other analytes (solutes) into sweat by a process of natural electro-osmosis. An embodiment of the disclosed invention relies on entry primarily through the cell-cell junctions rather than through additional electrically formed pores. Relying entirely on iontophoresis for fluid access with large sample volumes creates large pores and damages the paths through tissue and cells. Whether a majority of the porous pathways are natural or are created may be determined through the measurement of electrical impedance with the skin. Electrical impedance of the skin will increase with decreasing sweat rate as more sodium and chloride is captured by the dermal duct. At a constant sweat rate, it will decrease only if new porous pathways are created through or between plasma membranes of cells by an excess of current or voltage. The same can be true for interstitial fluid extracted without electroporation, because the dermal duct can recapture sodium and chloride at low generation rates for interstitial fluid in the same way it recaptures sodium and chloride for sweat. Exemplary voltages which will not electroporate a single cell plasma membrane are on the order of, but not limited to, 0.15 to 0.3 V, with electroporation typically being rapidly induced at 0.5 to 1 V across a single plasma membrane. The lining of the sweat gland has several cells, with at least two plasma membranes in series for the case of a single cell, such that an exemplary safe upper limit for applied reverse iontophoresis voltage without causing electroporation is 300 to 600 mV or less. For example, the reverse iontophoresis voltage could be ramped slowly or tested at several levels, and the skin impedance could be measured continuously or repeatedly. These measurements may be used to determine a safe level of voltage to avoid new plasma membrane poration to the point where the dominant entry of flux of analytes is due to new pores as opposed to natural pathways that existed before reverse iontophoresis was applied. To that end, an embodiment of the disclosed invention includes an electrode or sensor for measuring skin impedance. For example, with reference to FIG. 1A, the electrode 150 could be used both for reverse iontophoresis and for measuring skin impedance. In another embodiment, a skin impedance sensor is used to determine limits for the amount of reverse iontophoresis that is applied. Furthermore, to obtain less drift in the impedance due to skin hydration, changing skin contact, and other confounding factors, another embodiment may include a first electrode as a reference impedance sensor for measuring skin impedance in a first location where no iontophoresis is applied (not shown) and a second electrode as an impedance sensor for measuring skin impedance in a second location where iontophoresis is applied.

When new pore formation begins to occur, there is likely a clear non-linear change in skin impedance as the natural pathways will tend to behave more like a classical resistor in response to voltage (albeit not perfectly linear as they are biological structures), and the new pores will create a superlinear response (e.g., they can get bigger and more numerous over time and the impedance will increase above the expected linear line). An applied potential of only 0.5 V to the stratum corneum shows little or no change in electrical conductance over time, and application of 0.75 V and 1 V showed fairly good stability of conductance up to an hour or more. Therefore, in various embodiments, a reverse iontophoresis voltage of less than 3 V and preferably less than 1V may be applied. The predicted current density for an applied voltage of 1 V and is about 0.01 mA/cm², and, at 0.21 μL/mA/min, the predicted sample generation rate for the interstitial fluid is about 2.1 nL/min/cm² or 0.02 nL/min/gland for 100 glands/cm². However, this current density could still increase the analyte concentration coming in from interstitial fluid by 3× for sweat at 0.1 nL/min/gland sweat generation rate. If 3V were used, close to 10× higher concentration might be achieved.

With further reference to FIG. 1A, in an embodiment of the disclosed invention, a voltage drop across the biofluid could be sensed by the one or more of the sensors 120, 122, 124 and the reverse iontophoresis electrode 150. Because the sensors 120, 122, 124 and the reverse iontophoresis electrode 150 are in contact with the wicking collector 136, which contains electrically conductive biofluid during use, the sensors 120, 122, 124 and the electrode 150 will contact a fluid that could be at equipotential (same voltage). The voltage drop between the electrode 150 and the biofluid could then be used for feedback control of the voltage applied between electrodes 150 and 152, in order to ensure the voltage drop between electrode 150 and the biofluid remains below a level at which the pH would be significantly altered. Thus, the voltage applied to the iontophoresis electrode may be regulated using feedback by measuring the voltage drop between the iontophoresis electrode and biofluid. Such a voltage drop can be measured in several ways, including use of reference electrodes or sensors as known by those skilled in the art. For example, the sensor 120 could measure the voltage of the biofluid in the wicking collector 136. Therefore, the voltage drop between the electrode 150 and the biofluid may be determined along with the voltage of the electrode 150. In another embodiment, the voltage could be scanned at electrode 150 to determine when the pH is altered by measuring a change in the current response at electrode 150 (e.g., using cyclic voltammetry). In other words, a sensor may measure the voltage between the iontophoresis electrode and biofluid adjacent to the iontophoresis electrode.

In another aspect of the disclosed invention, reverse iontophoresis may be applied without causing significant electroporation by allowing adequate time for the skin to recover after voltage is removed or reduced. In that regard, if electroporation occurs, then a non-linear response may exist between the measured skin electrical impedance and increasing applied voltage (primarily electrical resistance), and/or the relationship between voltage and skin electrical resistance may change versus time even at constant voltage. The skin and/or tissue subjected to electroporation tends to heal, and the electrical resistance should recover over time if the voltage is removed. In an embodiment, a device may apply reverse iontophoresis for a period of 10 minutes for a given applied voltage, which if applied continuously would cause significant electroporation, but the device may then allow 50 minutes resting without reverse iontophoresis such that little or no accumulation of electroporation occurs. In another embodiment a device includes a sensor to measure the electrical resistance of the skin, and the application of the reverse iontophoresis could be regulated based on the measured electrical resistance to ensure excessive electroporation of the skin does not occur. For example, if DC voltage were applied for the reverse iontophoresis, then the DC current could also be measured to directly predict the total electrical resistance. For example, the reverse iontophoresis may be regulated to ensure that the electrical resistance of skin does not drop by more than 3× compared to the electrical resistance without reverse iontophoresis. In an embodiment, a first electrical resistance for skin with no iontophoresis and a second electrical resistance for skin with iontophoresis, where said first electrical resistance is no more than 3× greater than said second electrical resistance. This 3× would be in the context of unchanging skin conditions (e.g., start measuring impedance once the skin is fully hydrated or at a constant chemically stimulated sweat rate). One skilled in the art will recognize that embodiments of the disclosed invention may account for variations with electrode distances, changes between users, changes during use for a single user, etc. The absolute voltage applied between electrodes is, at least in part, dependent on electrode distance and physiological factors.

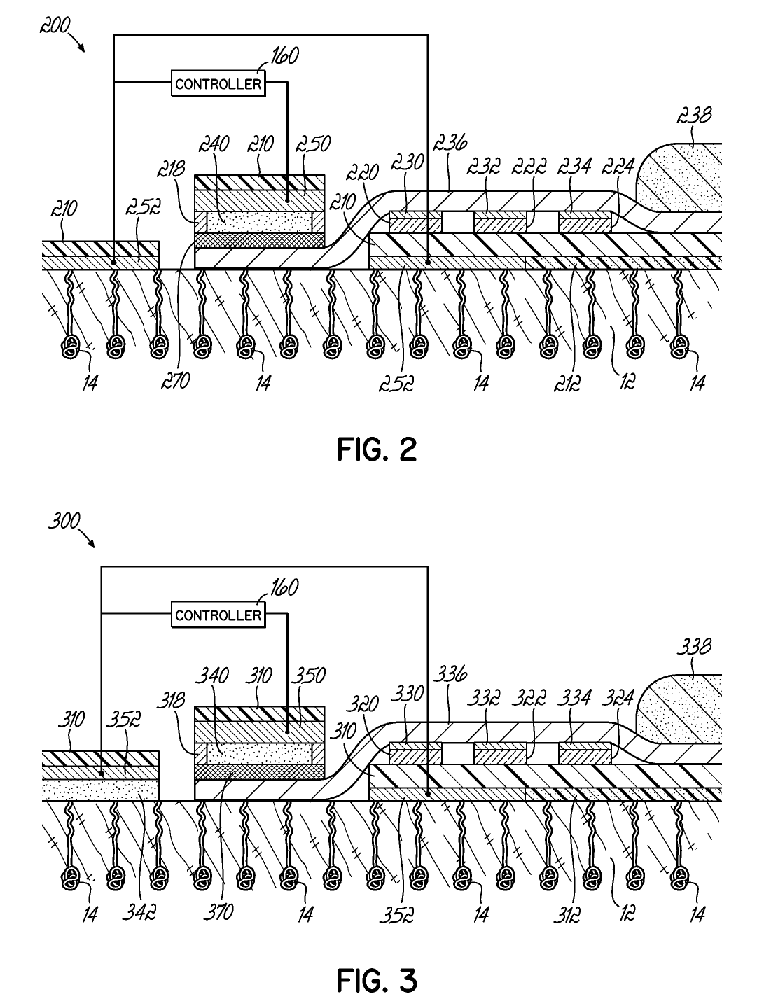

With reference to FIG. 2, in an embodiments of the disclosed invention, where like numerals refer to like features previously described for the device 100 (e.g., elements 220, 222, 224 are sensors specific to at least one analyte), a device 200 is capable of applying reverse iontophoresis with buffering of pH while also minimizing contamination of the sampled biofluid with pH or buffer or buffering by-products. The device 200 includes a permselective membrane 270 with a low porosity or selective porosity that is positioned between the iontophoresis electrode 250 the skin 12. As shown, there may be an intervening layer(s) between the permselective membrane 270 and the skin 12, such as the wicking collector 236. The permselective membrane 270 is a semipermeable membrane that is also an ion exchanger. The permselectivity could be to biofluid, to water, to chemicals, to analytes (e.g., size exclusion to proteins) or other aspects of fluids or solutes. In the case of a track-etch membrane, the permselectivity allows iontophoresis (ion-exchange) but substantially decreases advective flow or diffusion through the membrane. Exemplary materials for the permselective membrane 270 include a track-etch membrane, ultrafiltration membrane, ion-selective membrane, dialysis membrane, combinations thereof, or other type of membranes that separate generated pH or buffer or buffering byproducts from the sampled biofluid. The device 200 further contains a solution or gel, buffering solution, buffering material, or buffering gel 240, which is at least partially contained by the membrane 270, and a sealing wall, such as a polymer 218. The material 240 could act as a buffering component if it has an adequate volume simply by dilution of compounds that alter pH (i.e. physically buffering, rather than chemically buffering). The device 200 also includes a reverse iontophoresis electrode 250, which is carried by a substrate 210, a counter electrode 252, and a controller 160. When the wicking collector 236 is transporting biofluid towards the sensors 220, 222, 224, electrical current for the reverse iontophoresis could flow through the membrane 270, while the membrane 270 would significantly reduce or block the passive diffusion or advective flow of generated pH (acid or base) or of buffering agent or buffering byproducts. Because the skin 12 is highly electrically resistive and contains very few pores, it is not that difficult to obtain a porosity for the membrane 270 that is very low and which substantially reduces or blocks passive diffusion of many types of analytes while also allowing adequate electrical conductivity for reverse iontophoresis. Generally, the osmotic pressure may be balanced between the collected biofluid and the solution, gel, or material 240. To summarize, an embodiment of the disclosed invention may include at least one permselective membrane in between a reverse iontophoresis electrode and the wicking collector. A permselective membrane 270 could also be used in alternate embodiments that do not include a wicking collector (i.e., it includes a suitable substitute, such as a microfluidic channel that has a flow of sweat driven by the positive pressure of sweat, such as will be described for FIG. 7). In another embodiment (not shown), the membrane 270, sealing wall 218, and solution, gel, or material 250 could be utilized for the counter electrode 252. Therefore, an embodiment of the disclosed invention may include at least one permselective membrane between an iontophoresis electrode and skin.

With reference to FIG. 3, in an embodiment of the disclosed invention, where like numerals refer to like features previously described for the devices 100 and 200, a device 300 includes components 342, 354, which can be used for a variety of functions such as sweat stimulation, sweat suppression, numbing of skin, or reducing inflammation of the skin. For example, the element 354 could be an iontophoresis electrode, and the element 342 could be a hydrogel, such as agar, containing a chemical to be delivered to the skin 12, such as carbachol, atropine, or hydrocortisone. Because the dermis is typically several mm thick, if element 342 is within, for example, several hundred μm of the wicking collector 336, then a chemical originating from the element 342 can be iontophoretically driven or diffused horizontally into the skin 12 by the electrode 354 beneath where the wicking collector 336 contacts skin 12. Although the exemplary method described herein for chemical delivery is by iontophoresis, any suitable delivery method by diffusion, injection, with the use of skin permeability enhancers, or other techniques are included within the scope of the disclosed invention. Furthermore, chemicals described herein could also be included in other suitable components for delivery to the skin 21, such as the solution, gel, or material 340. The chemical containing element 342 could also be separated from skin 12 and/or a wicking collector component 336 (not shown in contact with the element 342) using a permselective membrane.

Exemplary sweat stimulants include acetylcholine, pilocarpine, methacholine, and carbachol, among others. The sweat stimulation mechanism may be used to initiate sweating to establish a reduced volume pathway and/or electrical connection between a reverse iontophoresis electrode and pre-existing pathways. In an embodiment, the sweat stimulant has a sweat stimulation duration of less than 60 minutes and, after sweat stimulation, reverse iontophoresis is applied to extract interstitial fluid. In that respect, acetylcholine is rapidly metabolized by the body, and sweating would stop occurring even within several minutes. This would allow the device 300 to be quickly primed with biofluid, and, by using the reverse iontophoresis electrode 350, then be able to sample interstitial fluid without dilution from sweat (after the sweating ceases). Some embodiments of the disclosed invention may apply reverse iontophoresis periodically rather than continuously (as described above), and a lack of iontophoresis for a period of time (e.g., minutes to hours) could cause the volume reduced pathway to disconnect or terminate fluidically or electrically. Therefore, the temporary stimulation of sweat, or instead stimulation of a constant low flow rate of sweat (e.g., less than 0.1 nL/min/gland) may be helpful as needed to maintain the volume reduced pathway and/or electrical connection for reverse iontophoresis. In other words, an embodiment of the disclosed invention includes the sampling of both stimulated sweat and interstitial fluid generated by reverse iontophoresis.

With further reference to FIG. 3, in an embodiment of the disclosed invention, sweat suppressants may be used to limit or prevent dilution of interstitial fluid by sweat. Furthermore, the elements 342, 354, which are used for sweat stimulation, could be used also for sweat suppression or delivery of other chemicals such as numbing or anti-inflammatory agents. For example, a positively charged sweat stimulant and a negatively charged sweat suppressant could be used such that sweat stimulation could be provided with a positive voltage to electrode 354 to establish the reduced volume pathway, followed by inhibition of sweating with a negative voltage to electrode 354. The suppression of sweating could allow more rapid use of the device 300 with less dilute or undiluted interstitial fluid and, furthermore, allow the device to better function without natural sweat events that could dilute the interstitial fluid and confound the sensing. Exemplary sweat inhibiting substances include, but are not limited to: any anticholinergic agent, scopolamine (which can be delivered transdermally even by diffusion), glycopyrrolate, atropine, benzatropine, antimuscarinic agents, antinicotinic agents, etc. Diffusing sweat inhibiting substances could also be incorporated in one or more of the materials described herein, such as an adhesive 312. Whether or not sweat suppression is applied and is working could be assessed and controlled by measuring skin impedances (e.g., electrode 350), Na+ concentration (e.g., sensor 320), a flow of fluid using thermal mass flow sensors (e.g., sensor 322), or another indicator in the absence of reverse iontophoresis (i.e., without reverse iontophoresis, then any such flow or reduced impedance or indicator of sweat would therefore be due to sweat). Therefore, an embodiment of the disclosed invention may include at least one sweat sensor that is in communication with at least one element for delivery of a sweat inhibiting substance.

With further reference to FIG. 3, in an embodiment of the disclosed invention, numbing or anti-inflammatory agents can be delivered using elements 342, 354, as described above, to mitigate discomfort, pain, inflammation, or other adverse effects caused by reverse iontophoresis. Again, such agents could be delivered by passive diffusion or charged and delivered by iontophoresis. Several non-limiting examples of numbing or anti-inflammatory agents include dexamethasone, hydrocortisone, salicylate, and lidocaine.

In an aspect of the disclosed invention, sweating that occurs during extraction of interstitial fluid could result in unknown dilution of analyte in the interstitial fluid, with exception to analytes that have sweat concentrations similar to those found in interstitial fluid (e.g., unbound concentrations of cortisol). An embodiment may include a sensor (e.g., measuring skin impedance, sodium concentration, or a thermal flow) to measure sweat generation, sweat sampling interval, and/or sweat flow rate in the absence of or during a pause from reverse iontophoresis. This measurement could then be used to determine the amount of dilution of interstitial fluid that occurs during reverse iontophoresis. Similarly, one or more sensors may be used to measure generation rate for interstitial fluid during reverse iontophoresis. For example, the composition of the interstitial fluid may be analyzed to determine if the fluid includes more or less than 50% sweat (i.e., the ratio of sweat to interstitial fluid in the biofluid). Both generation rates and/or flow rates of sweat, interstitial fluid, or the biofluid in general could be measured by at least one sensor. Further, another embodiment of the disclosed invention may include at least one sensor for determining the ratio of sweat to interstitial fluid in the biofluid (e.g., by methods such as measuring analyte dilution caused by sweat). Another embodiment of the disclosed invention may include at least one sensor for measuring at least one of sample generation rate or biofluid flow rate into the device (e.g., using a thermal flow meter).

In an embodiment of the disclosed invention where the sample volume is known and/or can be pre-determined, the above measures of generation rates and flow rates may also be used to provide chronological assurance of the sampling interval. With reference to FIG. 3, the series of sensors 330, 332, 334 could also measure flow rate, for example, by measuring the point when each sensor first receives a flow of biofluid that registers a change in analyte or pH concentration caused by reverse iontophoresis. If the wicking collector 336 dimensions are known and, therefore, the sweat volume is known above the sensors 330, 332, 334, then the sampling interval and biofluid flow rates can then be calculated. The sampling interval, or chronological assurance, could also be pre-set or programmed into a device 300. The sample generation rate could also be controlled by feedback control based on measurement of the actual sampling interval or chronological assurance. In other words, an embodiment may include a sensor for measuring a sampling interval, the sensor being in communication with an iontophoresis controller (e.g., controller 160 in FIG. 1A).

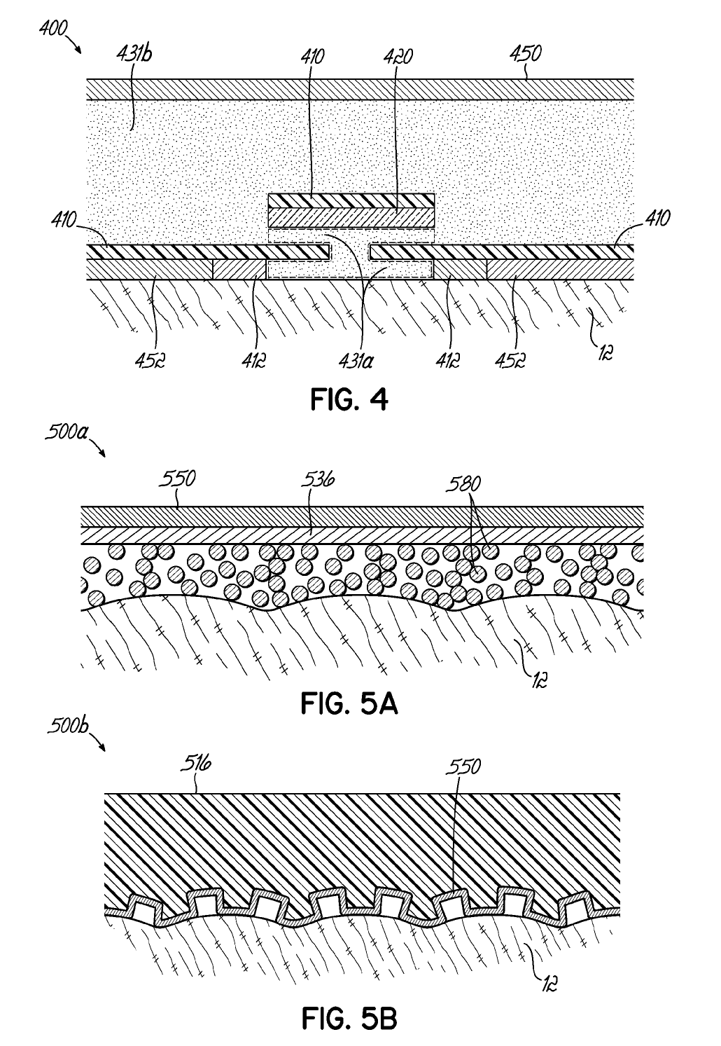

With reference to FIG. 4, in an embodiment of the disclosed invention, where like numerals refer to like features previously described for the device 100, a device 400 achieves a reduced sample volume using a different configuration compared to the device 100. Device 400 has a large-volume hydrogel 431. The area 431a encircled by the dashed line represents the portion of the overall volume that acts as the sample volume. Portion 431b is large enough (e.g., 1000 μm thick) to potentially mitigate pH issues during reverse iontophoresis. In other words, as the biofluid is transported through portion 431a, past the sensor 420, and into the portion 431b, the analytes in the biofluid that may affect the pH near the electrode 450 may be diluted due to the volume of the portion 431b. In an embodiment, the portion 431a could comprise a horizontal area that is similar to that of the sensor 420 that is 1×1 mm in area or about 0.01 cm² in area, have thickness of 15 μm in between sensor 420 and substrate 410, and have thickness of 30 μm on average between the substrate 410 and the skin 12. The sample volume would therefore be about 4E-5 cm³ or 40 nL. If one pre-existing pathway existed, assuming 100 glands/cm² and a sample generation rate of 4 nL/min for sweat and interstitial fluid, a sampling interval of 10 minutes could be achieved. Further, the sensor 420 has a centered flow of biofluid, which aids in minimizing the sampling interval.

With reference to FIG. 5A and 5B, in additional embodiments of the disclosed invention, where like numerals refer to like features previously described, the devices 500a and 500b are shown. At least a portion of the devices 500a and 500b contain a volume reducing material that is electrically conductive and conformal with skin (i.e., including hair, skin defects, debris, etc.). A volume reducing material that is electrically conductive is beneficial for maintaining electrical contact between a reverse iontophoresis electrode and pre-existing pathways. For example, in FIG. 5A, electrically conductive beads 580 are utilized to fill spaces and, therefore, reduce the total volume occupied compared to a single component having the same total volume. Exemplary electrically conductive beads include gold, silver, silver-chloride, conductive polymers, or other suitable materials. Further, the conductive beads could include a buffering material against changes in pH. In FIG. 5B, the polymer 516 could be a silicone rubber that is highly soft and compliant with a thin coating of gold 550 or conductive polymer, carbon, flexible conductor (e.g., nanowires in a polymer) or other suitable electrode material that promotes conformality with the skin 12 and that is adequately hydrophilic (e.g., gold, carbon coated with agar, etc.). To ensure conformality, pressure may be applied, as described for FIG. 7.

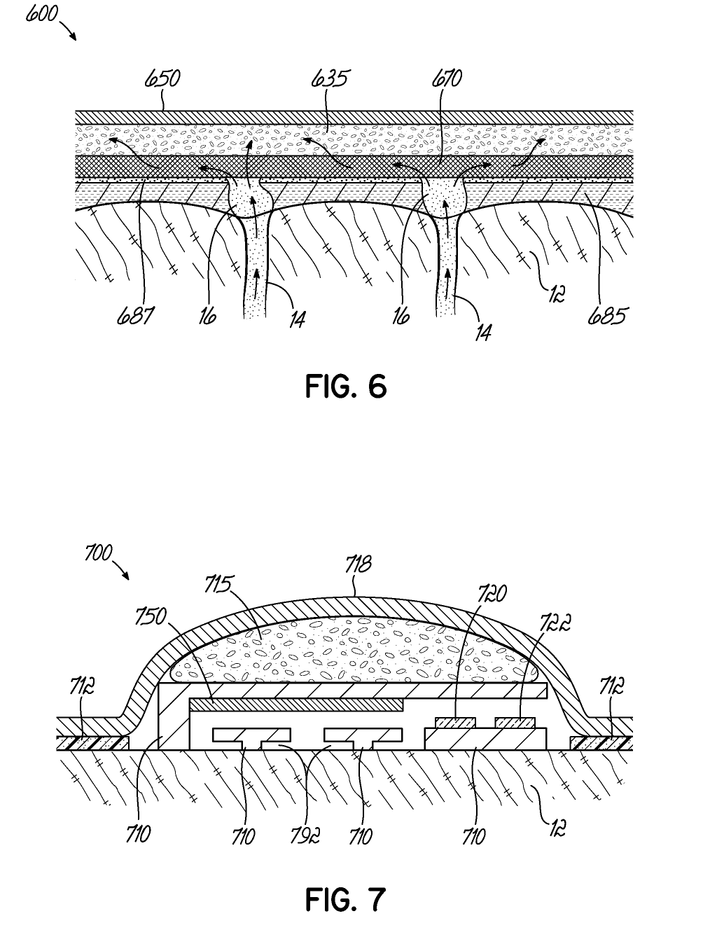

With reference to FIG. 6, in an embodiment of the disclosed invention, where like numerals refer to like features previously described, a device 600 achieves a reduced sample volume using an alternate configuration. In the device 600, sweat is used to establish at least a portion of a volume reduced pathway 16. The device 600 includes a biofluid impermeable and electrically insulating material 685 that could be, for example, a cosmetic oil, petroleum jelly, or other suitable material. The device 600 also includes a membrane 670 that is coated with, for example, a sweat dissolvable material 687. In an embodiment, the membrane 670 may be a hydrophilic track-etch membrane, and the sweat dissolvable material 687 may be poly-vinyl alcohol or sucrose that is 3 μm thick. The sweat dissolvable material 687 prevents the biofluid impermeable material 685 from fouling the sensors (not shown) or wicking materials, such as wicking material 635, which could be a textile. To begin forming the volume reduced pathway 16, the sweat dissolves the sweat dissolvable material 687, passes through the membrane 670, and moves into the wicking material 635. Once the sweat wets the wicking material 635, an electrical and fluidic connection is established between the sweat gland pre-existing pathways 14 and the reverse iontophoresis electrode 650. Therefore, even after sweat ceases to be generated, fluidic and electrical connection is maintained by the flow of biofluid and allows continued reverse iontophoresis and sample generation. Sweat stimulation could be natural or achieved using other methods as taught herein to activate the initial pathway between the reverse iontophoresis electrode 650 and pre-existing pathways 14.

Because sample volumes in embodiments of the disclosed invention are dramatically reduced, which allows for use with very low sample generation rates, a possible confounding factor is therefore transepidermal water loss. This effect is more than just a one-way transfer of water from skin to outside of the body, and depending on osmolality (which has greater osmolality, skin or collected biofluid), the collected sample of biofluid could either be concentrated (water loss) or diluted (water gain). FIG. 6 also shows a barrier on skin (e.g., oil or other material) that would block transfer of water between the skin 12 and areas of the device 600 that collect or transport bio fluid. Thus, an embodiment of the disclosed invention may include: a volume reducing material that is electrically insulating and conformal with skin; a water impermeable material that isolates pre-existing pathways from the rest of the skin surface; and a sweat stimulation element that establishes an electrically conductive and fluid conductive pathway through an electrically insulating volume reducing material.

With further reference to FIG. 6, during application of reverse iontophoresis, it is possible that an electrically insulating material (e.g., an oil or gas) eliminates or prevents electrical conduction between the reverse iontophoresis electrode 650 and the skin 12. As a result, a controller (not shown) for the reverse iontophoresis electrode 650 might raise the applied voltage to a large voltage to generate current, perhaps even through electrical breakdown of material 685, which could be painful or damaging to the skin 12. Therefore, a sensor could be used to determine when it is safe to apply reverse iontophoresis. For example, the electrode 650 could be used to sense the electrical impedance with skin. Once an electrically conductive and fluid conductive pathway is established, the electrical impedance would decrease significantly (as much as orders of magnitude or more). In other words, the sensor may be used to determine if a volume reduced pathway is formed that originates from pre-existing pathways.

With reference to FIG. 7, in an embodiment of the disclosed invention, where like numerals refer to like features previously described, a device 700 includes a memory foam 715 and stretchy protective textile 718 that are used to provide pressure on the device 700 when it is placed against the skin 12. Device 700 further includes a biofluid collector 710 pressed against the skin 12 containing sensors 720, 722 and having a plurality of pores or pathways. Because of the applied pressure, a reduced sample volume is achieved in the space between the skin 12 and the biofluid collector 710 compared to if the biofluid collector 710 was not pressed against the skin 12. Exemplary pressure elements include one or more of the following: an adhesive; a mechanical clamp; a spring; a strap; a plastic housing; a vacuum providing component; a suction providing component, or other suitable pressure elements. A pressure element may include a cushioning element, such as a sponge, memory foam, a fluid filled bag, a gel, or a hydrogel. In various embodiments, the device 700 could be pre-loaded with a conductive fluid to enable reverse iontophoresis or a establish a fluid pathway between reverse iontophoresis electrode 750 and pre-existing pathways by virtue of sweat or other methods as taught herein. The biofluid collector 710 may include a closed-cell network of pathways 792 that increase the open area of the pores in the biofluid collector 710 against the skin surface. Numerous methods could be used to achieve such pathways, including use of porous membranes, textiles, microchannels (as shown in FIG. 7), or other suitable materials or features that help form a volume-reduced pathway. Skin deformation varies from person to person and based on measurement location and skin hydration level as well. Generally, a pressure of 5,000-30,000 N/m² should yield a mechanical deformation between 0.6 to 1.6 mm of skin deformation under direct compression. In an embodiment of the disclosed invention, about 100 μm of indentation/deformation may be provided. An experimentally measured value of about 100 μm can be achieved within 15 minutes of pressure ranging from 600 to 4,000 N/m². In embodiments of the disclosed invention, a device may be applied with a pressure range for the sweat collector 110 against skin of 60 to 40,000 N/m², at least 60 N/m², at least 600 N/m², at least 4,000 N/m², or at least 40,000 N/m². A calculated maximum pressure that could occlude a highly active sweat gland is 70,000 N/m² for 15 nL/min/gland. Therefore, at lower sweat rates, lower applied pressures may be utilized because there is less hydraulic pressure created by the sweat glands. The applied pressure may be designed to avoid any issue with long-term pressure against skin that creates skin damage or issues with blood flow. Further, embodiments of the disclosed invention may include a plurality of pressure providing components that are used in combination with each other (e.g., a strap and a vacuum, or a plastic housing and a vacuum, or a clamp, a memory foam component and a strap, etc.). To allow reliable pressure in an embodiment where an adhesive 712 is used for holding the device 700 against skin 12, the contact area of the adhesive with skin 12 should be at least 3× greater than the contact area of the biofluid collector pressed against skin and, more preferably, 10× greater.

With reference to FIGS. 8A and 8B, in other embodiments of the disclosed invention, where like numerals refer to like features previously described , devices 800a and 800b each include sample generation components 880, 882 that can generate a flow of interstitial fluid, sweat, or both. Therefore, in FIG. 8A, the device 800a includes a sensor 820 that receives the biofluid sample from two different sample generation locations (i.e., near elements 880 and 882). Some analytes, such as cortisol, are best sampled rapidly through sweat generation, while some larger analytes, such as IL-6, are likely best sampled through interstitial fluid. Therefore, alternately, in FIG. 8B, the device 800b includes sensors 820, 822 that each have their own respective sample generation component 880, 882. Also illustrated in FIGS. 8A and 8B, the sample generation components 880, 882 can share a pump 838, a wicking collector 836, or the sensor 820 (FIG. 8A only). Therefore, an embodiment the disclosed invention may further include a plurality of sample generation components with a reduced sample volume for each.

The following examples are provided to help illustrate the disclosed invention, and are not comprehensive or limiting in any manner.

### EXAMPLE 1

Sweating was stimulated using the Wescor Nanoduct iontophoresis protocol with carabachol substituted for pilocarpine (0.5 mA for 1.3 mA-min, area stimulated was on the forearm with a 1.89 cm² disk). After the forearm stimulation site was left to sweat for 15 minutes, reverse iontophoresis was performed on half of the stimulated area using an ActivaDose controller set to apply 0.2 mA for 10 minutes. The active electrode was half of a 3% agarose disk within a custom holder. After 2 minutes, a 7% suspension of bromophenol blue in cosmetic-grade PDMS oil was applied to the skin to visualize sweating. The voltage applied during reverse iontophoresis was largely constant during most of the test. This experiment was promising as it showed no detectable reduction in sweat rate due to iontophoresis. The sweat stimulation lasted for greater than 24 hours, which is greater than the 1-2 hours expected when using pilocarpine at practical doses (simply increasing the dose cannot provide longer stimulation with pilocarpine because it is rapidly metabolized). Accordingly, with lower doses of carbachol, sweat stimulation may last for more than 3 hours, more than 6 hours, more than 12 hours, or more than 24 hours.

### EXAMPLE 2

With reference to FIG. 1A, assuming the device 100 has a 10 mm² sample collection area on skin with 100 glands/cm² and the sample collector has 5 μm deep channels that comprise 5% of the surface area of the wicking component, the result is an biofluid sample volume of at least 2.5 nL (i.e., 5E-4 cm*0.05*0.1 cm²=2.5E-6 mL or 2.5 nL). The area of collection could be reduced even further to 3 mm² covering three pre-existing pathways for a volume of less than 1 nL. Assume the remainder of the wicking collector is negligible in volume or in impacting sampling interval (e.g., a thin strip leading over 50 μm wide sensors). If 10 pre-existing pathways with sample generation rates of 0.3 and 0.03 nL/min/gland are covered by the 10 mm² sample collection area (i.e., 3 and 0.3 nL/min, respectively), then the fastest sampling interval that the wicking component could enable would be about 0.8 to 8 minutes (i.e., reduced volume/sample volume per minute). In various embodiments of the disclosed invention, the sampling interval for an advective flow of interstitial fluid alone can therefore be faster than 60 minutes, faster than 30 minutes, faster than 15 minutes, faster than 5 minutes, and faster than 2 minutes. The sampling interval for sweat could be even faster with sweat generation rates exceeding 1 nL/min/gland. Also, therefore sample volumes and volume reduced pathways may be less than 1000 nL, less than 500 nL, less than 100 nL, less than 30 nL, less than 15 nL, less than 5 nL, less than 2.5 nL, or even less than 1 nL.

Also, a faster sampling rate due to a reduced sample volume can be used to reduce the reverse iontophoresis current density. For example, consider a reverse iontophoresis current density of 0.3 mA/cm² where the sample volume is not reduced, and then an embodiment of the disclosed invention that reduces the sample volume by 500× may use a current density of 0.0006 mA/cm². This is a first order calculation that assumes advective flow rate of interstitial fluid is proportional to reverse iontophoresis current density. It is recognized that there will be individual variances in current densities used based on the intended application, and there may be a threshold current density that is too low to support a net advective flow toward the sensors. In some cases, sweat generation may provide the needed advective flow toward the sensors. In various embodiments of the disclosed invention, devices may operate with reverse iontophoresis current densities of less than 0.1 mA/cm², less than 0.05 mA/cm², less than 0.02 mA/cm², less than 0.01 mA/cm², less than 0.005 mA/cm², or less than 0.002 mA/cm². Further, because interstitial fluid can have a lag-time compared to blood, applying a device according to an embodiment of the disclosed invention where the dominant pre-existing pathway for analyte extraction is the sweat ducts may have a reduced lag-time compared to another dominant pre-existing pathway because the sweat glands are at least partially closely surrounded in some cases by a capillary bed with blood flow.

### EXAMPLE 3

Example 3 provides a hypothetical calculation of wicking pressures for elements of the disclosed invention. For purposes of the calculation, the wicking coupler will have the greatest wicking pressure, followed by the wicking pump, and lastly the wicking collector. These relative wicking pressure strengths will ensure that biofluid is continuously removed from the wicking collector so that negligible biofluid remains on the skin surface.

In all of the calculations, the wicking pressures originate from negative Laplace pressure, Δp=γ(1/R₁+1/R₂), where the surface tension of the biofluid is close to that of pure water (γ of about 70 mN/m). For simplicity, assume the fluid is constant, and principal radii, R₁ and R₂, are concave (negative). To further simplify the discussion, the effective radii for each sub-component is calculated (i.e., no need to quantify wicking pressure, smaller radii equals larger wicking pressure).

The space between skin and a wicking collector. First is a rough 2D calculation of what is required to reduce the effective biofluid volume to at least 10% of the available biofluid volume, which will also reduce skin surface contamination. Assume 60 μm peak-to-valley skin roughness, which would be greater with hair or skin defects. If the wicking collector is touching the skin, to reduce the effective biofluid volume to 10% of available volume, the biofluid will be wicked into a space that extends only 20 μm out from the skin ridges (assuming triangular ridge shapes), with a meniscus between skin and the collector that spans about 20 μm. Next, assume a biofluid contact angle on skin of θₛₖᵢₙ=0°, which represents a hydrated and swollen skin surface (typical contact angles are around 90°). Using a wicking collector made of polyamide (nylon, PA46) that is hot-embossed with a network of rectangular channels similar to those shown in FIG. 1B, with an averaged contact angle of θ_{poly}=45°. The wicking pressure for a long triangular groove in skin will be dominated by a single radius of curvature (Rₛₖᵢₙ), which can be visualized along the meniscus edge, and calculated as Rₛₖᵢₙ=−h/(cos[θ_{poly}−45°]+cos θₛₖᵢₙ)≅10 μm where h is about 20 μm as previously discussed and the “−45°” term denotes a converging capillary feature. Having calculated Rₛₖᵢₙ at about 10 μm, the wicking pressure required from the wicking collector can be determined.

The wicking collector. Assume the wicking collector has square cross-section microchannels with a 1:1 aspect ratio and width w, for which an effective single capillary radius, R_{collector}, can be calculated as R_{collector}=w/(3 cos(θ_{poly})−1). The wicking pressure of the collector may sustain the less than 10% biofluid volume between skin and the wicking collector, and therefore R_{collector}=Rₛₖᵢₙ=10 μm. This R_{collector} value yields a calculated channel width of about 11-12 μm. A suitable material for the wicking collector is polyamide (nylon), because it is easily microreplicatable, hydrophilic, and relative to many other polymers, exhibits lower non-specific biofluid protein and analyte binding. The wicking collector could be initially be coated with a layer of poly-vinyl-alcohol (PVA) water-dissolvable polymer of 10's of nm thickness, to enable wetting past channel junctions.

The wicking coupler. Next, assume a 10's of μm thick wicking coupler between the wicking collector and a sensor. For device operation, the wicking coupler must keep the sensors continually wetted with a new sample of biofluid. To achieve this, there is at least a 10× decrease in effective capillary radius or R_{coupler} about 1 μm (this includes a margin of error to allow for possible variances). There are several materials available from which a wicking material with micrometer-scale capillaries may be fabricated, for example, a hydrophilized nano-cellulose material that is 20 μm thick when hydrated. Nano-cellulose forms a gel-like material which remains cohesive even when hydrated due to microfibril interactions. Nano-cellulose is soft and should promote wetting to sensors. Another attractive possibility is to coat and polymerize a thin film of a hydrogel or super-porous hydrogel, or coating with agar. Hydrated hydrogels can have pore sizes sufficient to allow advective transport of even large proteins. Super-porous hydrogels have a physically open porous network that can be tuned from sizes of 100's of nm to several μm's. A hydrogel wicking coupler has further advantages because hydrogels (1) are pliant when wet and with slight pressure will remain in wetted contact with sensors; and (2) can be coated onto, and in some cases adhered to, the polyamide wicking collector or sensors.

The wicking pump. In this example, the pump serves primarily as a method to collect and dispose of excess biofluid throughout device operation. The wicking pump may have greater wicking pressure than the wicking collector, but its wicking pressure may not exceed that of the wicking coupler or the pump will remove biofluid from the wicking coupler and leave inadequate biofluid on the sensors for accurate measurements. The wicking pump having an effective wicking radius of Rₚᵤₘₚ=2-3 μm may be fabricated by simple techniques, such as stacking of a plurality of hydrophilic membrane filters (e.g., made of nitrocellulose or other membrane materials) that have well-tuned pore sizes and wicking pressure; or by use of relatively homogeneous beads (e.g., commercial monodisperse Reade Silica powder); a by use of a longer-chain length hydrogel; micro/nano-porous sponges; or other suitable components Again, the effective R_{coupler} could be decreased to 10's or 100's of nM to allow a wider selection of materials and effective radius Rₚᵤₘₚ for the wicking pump. The pump can be designed to store 10's to 100's of μL of biofluid, allowing for continuous use for greater than 24 hours at 0.5 nL/min/gland and 100 glands/cm² which is greater than 12 hours of continuous use, which is greater than 6 hours of continuous use. Note, the 10% volume between skin and the wicking collector could be further reduced by the wicking pressure of the wicking pump.

### EXAMPLE 4

Consider a device similar to the device in Example 2 with an effective space between skin and the wicking collector of 50 μm in height. If the wicking collector, wicking pump, and wicking coupler have greater wicking pressure than the wicking space, then the wicking space would be filled with biofluid. The approximate time to refresh this volume with new biofluid can be translated into biofluid sampling interval, and using first order calculations of simply refilling that volume, the sampling interval would be very long. If a wicking collector or other elements, like a wicking pump, are added to reduce or eliminate the sample volume associated with the effective 50 μm of space between skin and the collector, then the wicking collector should have an effective sample volume of less than 50 μm in the area that it is on or adjacent to skin. Otherwise, adding the wicking collector increases the total sample volume, meaning it does not help reduce the sample volume between the device and skin.

What is claimed is:

1. A device for sensing biofluid placed on skin adapted to cover at least one pre-existing pathway comprising:
a first analyte-specific sensor for sensing a first analyte in said biofluid, wherein said first analyte-specific sensor does not consume the first analyte;
a volume-reduced pathway between skin and said a first analyte-specific sensor configured to allow an advective flow of said biofluid from said at least one pre-existing pathway toward said first analyte-specific sensor; and
an iontophoresis electrode and a counter electrode adapted to cause said first analyte to move into said at least one pre-existing pathway.

2. The device of claim 1, wherein the biofluid is more than 50% interstitial fluid.

3. The device of claim 1, wherein the biofluid is more than 50% sweat.

4. The device of claim 1, further comprising a second analyte-specific sensor for a second analyte, wherein said first analyte and said second analyte have similar dilutions in said biofluid.

5. The device of claim 4, further comprising a controller configured to compare ratios of the first and second analytes over time.

6. The device of claim 1, wherein said first analyte-specific sensor has an area of less than 1 mm², 0.1 mm², 0.01 mm², or 0.001 mm².

7. The device of claim 1, further comprising at least one of: a wicking collector, a wicking coupler, or a wicking pump.

8. The device of claim 7, wherein said device comprises said wicking collector which is electrically conductive between said iontophoresis electrode and said at least one pre-existing pathway.

9. The device of claim 7, wherein said device comprises said wicking collector which has a greater wicking pressure than a wicking space between skin and said wicking collector.

10. The device of claim 7, wherein said device comprises said wicking collector and said wicking pump, said wicking collector having a wicking pressure greater than or equal to a wicking pressure of said wicking pump.

11. The device of claim 7, wherein said device comprises said wicking collector which has a sample volume that is less than a sample volume of a wicking space between said wicking collector and said skin.

12. The device of claim 7, wherein said device comprises said wicking collector, said wicking coupler, and said wicking pump, said wicking coupler having a wicking pressure that is greater than or equal to at least one of a wicking pressure of said wicking collector or a wicking pressure of said wicking pump.

13. The device of claim 7, wherein said device comprises said wicking collector and said wicking pump, said wicking pump having a greater wicking pressure than a wicking space between skin and said wicking collector.

14. The device of claim 1, further comprising a permselective membrane between said iontophoresis electrode and said skin.

15. The device of claim 14, further comprising a wicking collector between said permselective membrane and said skin.

16. The device of claim 14, further comprising a chemical containing element and a permselective membrane, wherein said permselective membrane is between said chemical containing element and said skin.

17. The device of claim 1, wherein said first analyte-specific sensor senses the first analyte in a first biofluid that is more than 50% sweat, the device further comprising a second analyte-specific sensor for sensing a second analyte in a second biofluid that is more than 50% interstitial fluid.

18. The device of claim 1, wherein the device applied iontophoresis less than 25%, less than 10%, or less than 5% of a total time of use of said device.

19. The device of claim 1, further comprising a controller for iontophoresis that activates iontophoresis on-demand, at set times, or at a time determined by a device user.

20. The device of claim 19, wherein said controller for iontophoresis is in communication with said least first analyte-specific sensor.

21. The device of claim 1, further comprising a pH sensor adapted to detect and correct for pH induced changes in said biofluid that is sensed by said first analyte-specific sensor.

22. The device of claim 1, further comprising a pH sensor adapted to determine a limit for the amount of iontophoresis that is applied.

23. The device of claim 1, wherein at least one of said iontophoresis electrode or said counter electrode is at least partially comprises a buffering material.

24. The device of claim 1, wherein at least one of said iontophoresis electrode or said counter electrode is at least partially coated with a buffering material.

25. The device of claim 1, further comprising a buffering agent for regulating pH.

26. The device of claim 1, wherein said iontophoresis electrode and said counter electrode differ in area by at least 2×, at least 10×, or at least 20×.

27. The device of claim 1, wherein said device has an area of electrical contact with skin for iontophoresis, and wherein said iontophoresis electrode has a contact area with skin that is at least 2 times greater than said area of electrical contact of said device with skin.

28. The device of claim 1, wherein said device has an area of electrical contact with skin for iontophoresis that is less than 0.5 cm², 0.25 cm², 0.1 cm², 0.05 cm², or 0.025 cm².

29. The device of claim 1, wherein said first analyte-specific sensor has a sensor area that is at least less than 0.001 mm², less than 0.01 mm², less than 0.1 mm², or less than 1 mm².

30. The device of claim 1, further comprising a sensor for measuring skin impedance.

31. The device of claim 30, further comprising a controller adapted to determine a limit for the amount of iontophoresis that is applied using a measurement of skin impedance.

32. The device of claim 1, further comprising a first electrode as a reference impedance sensor for measuring skin impedance in a first location where iontophoresis is not applied, and a second electrode as an impedance sensor for measuring skin impedance in a second location where iontophoresis is applied.

33. The device of claim 1, wherein, when iontophoresis is applied, said iontophoresis electrode provides an iontophoresis voltage that is less than 3V or less than 1V.

34. The device of claim 1, further comprising a sensor for measuring a voltage between said iontophoresis electrode and a biofluid sample that is adjacent to said iontophoresis electrode.

35. The device of claim 32, further comprising a first sensor for measuring a first electrical resistance for skin in said first location, and a second sensor for measuring a second electrical resistance for skin in said second location, wherein said first electrical resistance is less than 3× greater than said second electrical resistance.

36. The device of claim 1, further comprising a wicking collector at least partially comprising a network of wicking pathways adjacent to said skin.

37. The device of claim 36, wherein said network of wicking pathways comprise less than 50%, less than 30%, less than 20%, or less than 10% of an available horizontal surface area of said network of wicking pathways adjacent to said skin.

38. The device of claim 1, wherein said iontophoresis electrode has a first potential and said first analyte specific sensor has a second potential and said first and second potentials are the same during iontophoresis.

39. The device of claim 1, further comprising a wicking material where a sample volume in said wicking material during device use is less than 50% of a total available volume of said wicking material.

40. The device of claim 1, further comprising at least one of a component for sweat stimulation; a component for sweat suppression; a component for numbing the skin; or a component for reducing inflammation of the skin.

41. The device of claim 40, further comprising a sweat sensor that is in communication with said component for sweat suppression.

42. The device of claim 1, further comprising a chemical containing element and a permselective membrane, wherein said permselective membrane is between said chemical containing element and said skin.

43. The device of claim 40, wherein said component for sweat stimulation is capable of causing sweating for a duration of less than 60 minutes.

44. The device of claim 1, further comprising a sensor for measuring at least one of: a sweat flow rate or a sweat generation rate.

45. The device of claim 1, further comprising a sensor for determining a ratio of sweat to interstitial fluid in the biofluid.

46. The device of claim 1, further comprising a sensor for measuring at least one of a: biofluid flow rate or a biofluid generation rate.

47. The device of claim 1, further comprising a sensor for measuring at least one of: a chronological assurance or a sampling interval.

48. The device of claim 1, further comprising a sensor for determining a sampling interval and an iontophoresis controller, wherein said sensor for determining the sampling interval is in communication with said iontophoresis controller.

49. The device of claim 1, further comprising a volume reducing material that is electrically conductive with skin and is conformable with skin.

50. The device of claim 1, further comprising a volume reducing material that is electrically insulating and conformable with skin.

51. The device of claim 50, further comprising a sweat stimulation component that establishes an electrically conductive and fluidically conductive pathway through said volume reducing material.

52. The device of claim 1, further comprising a water impermeable material that isolates said at least one pre-existing pathway from the rest of the skin surface.

53. The device of claim 1, further comprising a sensor for determining at least one of the following: whether iontophoresis may be applied; or whether a volume-reduce pathway is formed that originates from said at least one pre-existing pathway.

54. The device of claim 1, further comprising:
a reduced sample volume;
a biofluid collector adapted to be placed on or adjacent to skin including a plurality of pores or pathways for entry of biofluid from skin into said biofluid collector; and
a pressure element capable of holding said biofluid collector against skin with a pressure and forming a volume-reduced pathway in a space between said biofluid collector and skin.

55. The device of claim 54, wherein said pressure is at least 60 N/m² or at least 600 N/m².

56. The device of claim 54, wherein said pressure less than 40,000 N/m² or less than 4,000 N/m².

57. The device of claim 54, wherein said pressure element comprises at least one of: an adhesive; a memory foam; a sponge; a mechanical clamp; a spring; a fluid filled bag; a gel; a hydrogel; a strap; a plastic housing; a vacuum providing component; or a negative fluid pressure providing component.

58. The device of claim 54, wherein said pressure element comprises an adhesive that has a contact area with skin that is 3 times greater or 10 times greater than a contact area of said biofluid collector with skin.

59. The device of claim 1, further comprising a first location for biofluid sample generation and a second location for biofluid sample generation.

60. The device of claim 59, wherein said first analyte-specific sensor receives biofluid from said first location for biofluid sample generation, the device further comprising:
a second analyte-specific sensor that receives biofluid from said second location for biofluid sample generation.

61. The device of claim 59, wherein said first location for biofluid sample generation and said second location for biofluid sample generation are fluidically connected to at least one of a: wicking pump, a wicking collector, or said first analyte-specific sensor.

62. The device of claim 1, further comprising a plurality of sample generation components each with a reduced sample volume.

63. The device of claim 1, further comprising a sweat stimulation component and a sweat stimulant chemical that is capable of providing continuous sweating for a duration of greater than 3 hours, greater than 6 hours, greater than 12 hours, or greater than 24 hours.

64. The device of claim 1, wherein a sample volume between said first analyte-specific sensor and pre-existing pathways is less than 1000 nL, less than 500 nL, less than 100 nL, less than 30 nL, less than 15 nL, less than 5 nL, less than 2.5 nL, or less than 1 nL.

65. The device of claim 7, wherein said wicking pump has a fluidic capacity and, at 0.5 nL/min/pathway and 100 pathways/cm², said capacity exceeds 6 hours of continuous use, 12 hours of continuous use, or 24 hours of continuous use.

66. A method of collecting and sensing biofluid comprising:
performing iontophoresis on at least one pre-existing pathway in skin;
receiving an advective flow of biofluid fluid through at least a portion of a volume reduced pathway between skin and a first analyte-specific sensor; and
sensing a first analyte in the biofluid using said first analyte-specific sensor.

67. The method of claim 66, further comprising sensing a second analyte in the biofluid using a second analyte-specific sensor, and comparing ratios of said first analyte and said second analyte over time.

68. The method of claim 66, further comprising establishing at least a portion of the volume reduced pathway using sweat.

69. The method of claim 66, further comprising stimulating sweating.

70. The method of claim 66, further comprising measuring a sweat sampling rate when iontophoresis is not being performed.

71. The method of claim 66, further comprising measuring an interstitial fluid sampling rate when there is no sweating from said skin.

72. The method of claim 66, further comprising controlling an applied current of the iontophoresis based on a measurement of an interstitial fluid sampling rate.

73. The method of claim 66, further comprising controlling a voltage applied for iontophoresis based on a measurement of a skin impedance.

74. The method of claim 73, wherein performing iontophoresis includes decreasing said skin impedance to a value that is no more than 3 times less than a value of a skin impedance without said iontophoresis.

75. The method of claim 66, wherein iontophoresis is applied on demand.

76. The method of claim 66, wherein iontophoresis is applied irregularly.

77. The method of claim 66, wherein sensing includes sensing two or more analytes, the method further comprising comparing results of the sensing of the two or more analytes.

78. The method of claim 66, further comprising reporting a chronological assurance that is not predetermined.

79. The method of claim 66, further comprising measuring sweat to determine when to perform iontophoresis.

80. The method of claim 66, further comprising regulating a voltage applied to cause iontophoresis using feedback control by measuring a voltage between the iontophoresis electrode and said biofluid that is in contact with said iontophoresis electrode.

81. The method of claim 66, wherein performing iontophoresis includes performing iontophoresis on pre-existing pathways that are more than 50% sweat glands.

82. The method of claim 66, wherein performing iontophoresis includes extracting a plurality of biofluid samples.

83. The method of claim 66, wherein performing iontophoresis includes applying a plurality of iontophoresis waveforms with individual durations of less than 10 ms.

84. The method of claim 66, wherein performing iontophoresis includes a ratio of a current density to a biofluid generation rate is less than 50 A/L/min.

85. The method of claim 66, wherein performing iontophoresis includes creating a current density on skin that is less than 0.1 mA/cm², less than 0.05 mA/cm², less than 0.05 mA/cm², less than 0.02 mA/cm², less than 0.01 mA/cm², less than 0.005 mA/cm², or less than 0.002 mA/cm².

86. The method of claim 66, wherein performing iontophoresis includes periodically reversing a polarity of said iontophoresis electrode.
